SEQUENCE LISTING (20 sequences)

SEQ ID NO: 1 - protein, 617 aa, Mus musculus
  misc_feature: VGF Full-length
MKTFTLPASVLFCFLLLIQGLGAAPPGRPDVFPPPLSSEHNGQVAEDAVSRPKDDGVPEVRAARNPEPQDQGELFQGVDPRALASVLLQALDRPASPPSVPGGSQQGTPEEAAEALLTESVRSQTHSLPAPEIQAPAVAPPRPQTQDRDPEEDDRSEELEALASLLQELRDFSPSNAKRQQETAAAETETRTHTLTRVNLESPGPERVWRASWGEFQARVPERAPLPPPVPSQFQARMSESAPLPETHQFGEGVSSPKTHLGETLTPLSKAYQSLGGPFPKVRRLEGSFLGGSEAGERLLQQGLAQVEAGRRQAEATRQAAAQEERLADLASDLLLQYLLQGGARQRDLGGRELQETQQERENEREEEAEQERRGGGEDDVGEEDEEAAEAEAEAEEAERARQNALLFAEEEDGEAGAEDKRSQEEAPGHRRKDAEGAEEGGEEDDDDEEMDPQTIDSLIELSTKLHLPADDVVSIIEEVEEKRKRKKNAPPEPVPPPRAAPAPTHVRSPQPPPPAPARDELPDWNEVLPPWDREEDEVFPPGPYHPFPNYIRPRTLQPPASSRRRHFHHALPPARHHPDLEAQARRAQEEADAEERRLQEQEELENYIEHVLLHRP

SEQ ID NO: 2 - protein, 615 aa, Homo sapiens
  misc_feature: VGF full-length
MKALRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAPESPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRPESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAEAEAEEAERARQNALLFAEEEDGEAGAEDKRSQEETPGHRRKEAEGTEEGGEEEDDEEMDPQTIDSLIELSTKLHLPADDVVSIIEEVEEKRKRKKNAPPEPVPPPRAAPAPTHVRSPQPPPPAPAPARDELPDWNEVLPPWDREEDEVYPPGPYHPFPNYIRPRTLQPPSALRRRHYHHALPPSRHYPGREAQARRAQEEAEAEERRLQEQEELENYIEHVLLRRP

SEQ ID NO: 3 - protein, 21 aa, Mus musculus
  misc_feature: TLQP-21Sequence
TLQPPASSRRRHFHHALPPAR

SEQ ID NO: 4 - protein, 21 aa, Homo sapiens
  misc_feature: Human TLQP-21Sequence
TLQPPSALRRRHYHHALPPSR

SEQ ID NO: 5 - protein, 62 aa, Mus musculus
  misc_feature: TLQP-62 Sequence
TLQPPASSRRRHFHHALPPARHHPDLEAQARRAQEEADAEERRLQEQEELENYIEHVLLHRP

SEQ ID NO: 6 - protein, 62 aa, Homo sapiens
  misc_feature: Human TLQP-62 Sequence
TLQPPSALRRRHYHHALPPSRHYPGREAQARRAQEEAEAEERRLQEQEELENYIEHVLLRRP

SEQ ID NO: 7 - protein, 30 aa, Mus musculus
  misc_feature: AQEE-30 Sequence
AQEERLADLASDLLLQYLLQGGARQRDLGG

SEQ ID NO: 8 - protein, 30 aa, Homo sapiens
  misc_feature: Human AQEE-30 Sequence
AQEERLADLASDLLLQYLLQGGARQRGLGG

SEQ ID NO: 9 - protein, 26 aa, Mus musculus
RLEGSFLGGSEAGERLLQQGLAQVEA

SEQ ID NO: 10 - protein, 26 aa, Homo sapiens
  misc_feature: Human NERP-1(PESA-25} Sequence
RPESALLGGSEAGERLLQQGLAQVEA

SEQ ID NO: 11 - protein, 38 aa, Mus musculus
  misc_feature: NERP-2 Sequence
QAEATRQAAAQEERLADLASDLLLQYLLQGGARQRDLG

SEQ ID NO: 12 - protein, 38 aa, Homo sapiens
  misc_feature: Human NERP-2 Sequence
QAEATRQAAAQEERLADLASDLLLQYLLQGGARQRGLG

SEQ ID NO: 13 - protein, 10 aa, Mus musculus
  misc_feature: LENY-10 sequence
LENYIEHVLL

SEQ ID NO: 14 - protein, 10 aa, Homo sapiens
  misc_feature: Human LENY-10 Sequence
LENYIEHVLL

SEQ ID NO: 15 - protein, 9 aa, Mus musculus
  misc_feature: RSQE-9 Sequence
RSQEEAPGH

SEQ ID NO: 16 - protein, 9 aa, Homo sapiens
  misc_feature: Human RSQE-9 Sequence
RSQEETPGH

SEQ ID NO: 17 - protein, 145 aa, Mus musculus
  misc_feature: VGF443-588
EEDDDDEEMDPQTIDSLIELSTKLHLPADDWSIIEEVEEKRKRKKNAPPEPVPPPRAAPAPTHVRSPQPPPPAPARDELPDWNEVLPPWDREEDEVFPPGPYHPFPNYIRPRTLQPPASSRRRHFHHALPPARHHPDLEAQARRA

SEQ ID NO: 18 - protein, 145 aa, Homo sapiens
  misc_feature: Human VGF443-588
DDEEMDPQTIDSLIELSTKLHLPADDWSIIEEVEEKRKRKKNAPPEPVPPPRAAPAPTHVRSPQPPPPAPAPARDELPDWNEVLPPWDREEDEVYPPGPYHPFPNYIRPRTLQPPSALRRRHYHHALPPSRHYPGREAQARRAQE

SEQ ID NO: 19 - protein, 237 aa, Mus musculus
  misc_feature: VGF4-240
FTLPASVLFCFLLLIQGLGAAPPGRPDVFPPPLSSEHNGQVAEDAVSRPKDDGVPEVRAARNPEPQDQGELFQGVDPRALASVLLQALDRPASPPSVPGGSQQGTPEEAAEALLTESVRSQTHSLPAPEIQAPAVAPPRPQTQDRDPEEDDRSEELEALASLLQELRDFSPSNAKRQQETAAAETETRTHTLTRVNLESPGPERVWRASWGEFQARVPERAPLPPPVPSQFQARMSE

SEQ ID NO: 20 - protein, 237 aa, Homo sapiens
  misc_feature: Human VGF4-240
LRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAPESPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGP

(19) United States
(12) Patent Application Publication
(10) Pub. No.: US 2017/0028021 A1
(43) Pub. Date: Feb. 2, 2017

(54) COMPOSITIONS AND METHODS FOR TREATING NEURODEGENERATIVE DISEASE

(71) Applicant: MYELIN THERAPEUTICS, INC., Brooklyn, NY (US)

(72) Inventor: Matias Alvarez-Saavedra, Santiago (CL)

(21) Appl. No.: 15/222,494

(22) Filed: Jul. 28, 2016

Related U.S. Application Data
(60) Provisional application No. 62/199,921, filed on Jul. 31, 2015.

(51) Int. Cl. A61K 38/18; A61K 48/00; C12N 7/00; G01N 33/68; C12N 15/86; A61K 38/46; A61K 9/00

(52) U.S. Cl. CPC A61K 38/18; A61K 38/465; A61K 48/0075; A61K 9/0085; G01N 33/6875; C12N 15/86; C12N 7/00; C12Y 301/00; A61K 48/00; C12N 2750/14143; G01N 2333/47

(57) ABSTRACT

The disclosure provides for compositions and methods for treating neurodegenerative and cardiovascular disease in a subject. The compositions and methods include delivering a nucleic acid molecule encoding VGF or a peptide thereof to a subject. The disclosure further provides methods for the diagnosis of neurodegenerative and cardiovascular disease.

### CROSS-REFERENCE TO RELATED APPLICATIONS

This application claims benefit of priority under 35 U.S.C. §119(e) of U.S. Ser. No. US 62/199,921 filed Jul. 31, 2015, the entire contents of which is incorporated herein by reference in its entirety.

### STATEMENT AS TO FEDERALLY SPONSORED RESEARCH

This invention was made with government support under Grant #MOP97764 and MOP84412 awarded by Canadian Institutes of Health Research (CIHR).

### INCORPORATION OF SEQUENCE LISTING

The material in the accompanying sequence listing is hereby incorporated by reference into this application. The accompanying sequence listing text file, name MYELIN1100_1_Sequence_Listing.txt, was created on Jul. 20, 2016, and is 24 kb. The file can be assessed using Microsoft Word.

### FIELD OF THE INVENTION

The invention relates generally to neurodegenerative diseases and more specifically to a method for treatment of neurodegenerative diseases by administering VGF to a subject in need thereof.

### BACKGROUND OF THE INVENTION

A neurodegenerative disease is a disease that results in the progressive loss of the structure and function of neurons, including neuronal death. The loss of neuronal function eventually leads to decreased or impaired brain function. Neurodegenerative diseases are challenging to treat and many of these diseases are currently incurable. Vascular growth factor (VGF) is a growth factor that plays roles in metabolism, homeostasis, and synaptic plasticity. It has been shown that neuropathic pain can be treated with VGF inhibitors. Secretion of VGF is increased in cerebrospinal fluid and blood in neurodegenerative disorders like Alzheimer's disease (AD) and VGF is a potential biomarker for these disorders. The disclosure herein provides compositions and methods for treating and diagnosing a neurodegenerative disease.

### SUMMARY OF THE INVENTION

The present invention is based on the seminal discovery that VGF is useful for the treatment of neurodegenerative diseases.

In one aspect, a method is provided for treating a neurodegenerative disease, comprising delivering to a subject suffering from the neurodegenerative disease a nucleic acid molecule encoding exogenous VGF or a peptide thereof, wherein the exogenous VGF or the peptide thereof is expressed in the subject. In some cases, the nucleic acid molecule is delivered by an adeno-associated viral (AAV) vector, a retroviral vector, or an adenoviral vector. In some cases, the AAV vector is derived from AAV-2 or AAV-9. In some cases, the subject is a human. In some cases, the delivering to a subject comprises intravenous, intranasal, intrathecal, or oral administration. In some cases, the delivering to a subject comprises intracranial administration. In a particular example, the exogenous VGF or peptide thereof is expressed in the brain of the subject. In some cases, the exogenous VGF or peptide thereof is expressed in glial cells. In some cases, the glial cells are oligodendrocytes or oligodendrocyte precursors. In some cases, the neurodegenerative disease is a demyelinating disease. In some cases, the demyelinating disease is cerebellar ataxia, multiple sclerosis, Alzheimer's disease, amyotrophic lateral sclerosis, or Friedreich's ataxia. In some cases, the treating results in remyelination, de novo myelination or hypermyelination. In some cases, the exogenous peptide is TLQP-21, TLQP-62, AQEE-30, NERP-1, NERP-2, LENY-10, RSQE-9,VGF₄₄₃₋₅₈₈ or VGF₄₋₂₄₀. In some cases, the exogenous VGF or peptide thereof is expressed at a higher level in the subject relative to the subject prior to delivery of the nucleic acid molecule. In some cases, the exogenous VGF or peptide thereof comprises at least 50% amino acid sequence homology to a native VGF or a peptide thereof. In some cases, the exogenous VGF or peptide thereof comprises at least 50% nucleic acid sequence homology to a native VGF or a peptide thereof In some cases, the native VGF is derived from a human. In some cases, the demyelinating disease is diagnosed by identifying at least one brain lesion in the subject by magnetic resonance imaging. In some cases, the subject has less brain lesions after the delivering than before the delivering.

In another aspect, a method is provided for treating a neurodegenerative disease, comprising delivering exogenous VGF protein or a peptide thereof to a subject suffering from the neurodegenerative disease. In some cases, the delivering comprises intravenous, intranasal, intrathecal or oral administration. In some cases, the neurodegenerative disease is a demyelinating disease. In some cases, the delivering comprises intracranial administration. In some cases, the demyelinating disease is cerebellar ataxia, multiple sclerosis, Alzheimer's disease, amyotrophic lateral sclerosis, or Friedreich's ataxia. In some cases, the exogenous peptide is TLQP-21, TLQP-62, AQEE-30, NERP-1, NERP-2, LENY-10, RSQE-9, VGF₄₄₃₋₅₈₈ or VGF₄₋₂₄₀. In some cases, the exogenous VGF protein or peptide thereof comprises at least 50% amino acid sequence homology to a native VGF protein or peptide thereof. In some cases, the native VGF protein is derived from a human. In some cases, the subject is a human. In some cases, the delivering results in remyelination, de novo myelination or hypermyelination. In some cases, the demyelinating disease is diagnosed by identifying at least one brain lesion in the subject by magnetic resonance imaging. In some cases, the subject has less brain lesions after the delivering than before the delivering.

In another aspect, a use of an exogenous VGF protein or a VGF peptide thereof to treat a neurodegenerative disease is provided. In some cases, the exogenous VGF protein comprises at least 50% amino acid sequence homology to a native VGF protein or a peptide thereof In some cases, the exogenous VGF protein or VGF peptide thereof is delivered to a subject. In some cases, the subject is a human. In some cases, the neurodegenerative disease is a demyelinating disease. In some cases, the demyelinating disease is cerebellar ataxia, multiple sclerosis, Alzheimer's disease, amyotrophic lateral sclerosis, or Friedreich's ataxia. In some cases, the exogenous VGF protein or VGF peptide results in remyelination, de novo myelination or hypermyelination. In some cases, the demyelinating disease is diagnosed by identifying at least one brain lesion in the subject by magnetic resonance imaging. In some cases, the subject has less brain lesions after the delivering than before the delivering.

In another aspect, a use of a nucleic acid encoding exogenous VGF protein or a VGF peptide thereof to treat a neurodegenerative disease is provided. In some cases, the exogenous VGF protein comprises at least 50% amino acid sequence homology to a native VGF protein or a peptide thereof. In some cases, the exogenous VGF protein or peptide thereof comprises at least 50% nucleic acid sequence homology to a native VGF protein or a peptide thereof. In some cases, the exogenous VGF protein or VGF peptide thereof is delivered to a subject. In some cases, the subject is a human. In some cases, the neurodegenerative disease is a demyelinating disease. In some cases, the demyelinating disease is cerebellar ataxia, multiple sclerosis, Alzheimer's disease, amyotrophic lateral sclerosis, or Friedreich's ataxia. In some cases, the exogenous VGF protein or VGF peptide results in remyelination, de novo myelination or hypermyelination. In some cases, the demyelinating disease is diagnosed by identifying at least one brain lesion in the subject by magnetic resonance imaging. In some cases, the subject has less brain lesions after the delivering than before the delivering.

In yet another aspect, a method is provided for diagnosing a neurodegenerative disease in a subject, the method comprising: a) detecting a level of VGF protein or a peptide thereof in a plasma sample of the subject; b) comparing the level of VGF protein or peptide thereof to that of a healthy control; and c) diagnosing the subject with the neurodegenerative disease if the level of VGF protein or peptide thereof is lower in the subject than the healthy control. In some cases, the diagnosing comprises diagnosing the subject with the neurodegenerative disease if the level of the VGF protein or VGF peptide thereof is at least 2-fold lower in the subject than the healthy control. In some cases, the diagnosing comprises diagnosing the subject with the neurodegenerative disease if the level of the VGF protein or VGF peptide thereof is at least 10-fold lower in the subject than the healthy control. In some cases, the detecting comprises mass spectrometry. In other cases, the detecting comprises an ELISA assay. In some cases, the neurodegenerative disease is a demyelinating disease. In some cases, the demyelinating disease is cerebellar ataxia, multiple sclerosis, Alzheimer's disease, amyotrophic lateral sclerosis, or Friedreich's ataxia. In some cases, the method further comprises administering to the subject a nucleic acid molecule encoding exogenous VGF or a peptide thereof, if the subject is diagnosed with the neurodegenerative disease. In other eases, the method further comprises administering to the subject an exogenous VGF protein or a peptide thereof, if the subject is diagnosed with the neurodegenerative disease.

In yet another aspect, a kit is provided comprising: a) a nucleic acid molecule encoding an exogenous VGF or a peptide thereof; and b) a means for delivering the nucleic acid molecule to a subject. In some cases, the nucleic acid molecule comprises a viral vector. In some cases, the viral vector is an adeno-associated (AAV) viral vector, an adenoviral vector or a retroviral vector. In some cases, the AAV vector is derived from AAV-2 or AAV-9. In some cases, the exogenous peptide thereof is TLQP-21, TLQP-62, AQEE-30, NERP-1, NERP-2, LENY-10, RSQE-9, VGF₄₄₃₋₅₈₈ or VGF₄₋₂₄₀. In some cases, the means for delivering comprises a needle.

In another aspect, a kit is provided comprising: a) an exogenous VGF or a peptide thereof; and b) a means for delivering the exogenous VGF or peptide thereof to a subject. In some cases, the exogenous peptide thereof is TLQP-21, TLQP-62, AQEE-30, NERP-1, NERP-2, LENY-10, RSQE-9, VGF₄₄₃₋₅₈₈ or VGF₄₋₂₄₀. In some cases, the means for delivering comprises a needle.

In yet another aspect, a method is provided for inducing remyelination, de novo myelination or hypermyelination in a subject, comprising administering (i) a nucleic acid molecule encoding an exogenous VGF or peptide thereof to a subject, wherein the exogenous VGF or peptide thereof is expressed in the subject, or (ii) an exogenous VGF protein or peptide thereof; wherein the administering induces remyelination, de novo myelination or hypermyelination in the subject. In some cases, the subject is suffering from a neurodegenerative disease. In some cases, the neurodegenerative disease is a demyelinating disease. In some cases, the demyelinating disease is cerebellar ataxia, multiple sclerosis, Alzheimer's disease, amyotrophic lateral sclerosis, or Friedreich's ataxia. In some cases, the demyelinating disease is diagnosed by identifying at least one brain lesion in the subject by magnetic resonance imaging. In some cases, the subject has less brain lesions after the administering than before the administering. In some cases, the exogenous VGF or peptide thereof is expressed in the brain. In some cases, the exogenous VGF or peptide thereof is expressed in glial cells. In some cases, the glial cells are oligodendrocytes or oligodendrocyte precursors.

In another aspect, a method is provided for treating a cardiovascular disease comprising delivering to a subject suffering from the cardiovascular disease a nucleic acid molecule encoding exogenous VGF or a peptide thereof, wherein the exogenous VGF or peptide thereof is expressed in the subject. In some cases, the nucleic acid molecule is delivered by an adeno-associated viral (AAV) vector, a retroviral vector, or an adenoviral vector. In some cases, the AAV vector is derived from AAV-2 or AAV-9. In some cases, the subject is a human. In some cases, the delivering to a subject comprises intravenous, intranasal, intrathecal, or oral administration. In some cases, the exogenous VGF or peptide thereof is expressed in cardiomyocytes. In some cases, the cardiovascular disease is rheumatic heart disease, valvular heart disease, aneurysm, atherosclerosis, hypertension, peripheral arterial disease, angina, coronary artery disease, coronary heart disease, myocardial infarction, stroke, cerebral vascular disease, transient ischemic attacks, cardiomyopathy, pericardial disease, congenital heart disease, heart failure, atrial fibrillation, endocarditis, aortic aneurysm, renal artery stenosis, myocarditis, or cardiomegaly. In some cases, the exogenous peptide is TLQP-21, TLQP-62, AQEE-30, NERP-1, NERP-2, LENY-10, RSQE-9, VGF₄₄₃₋₅₈₈, or VGF₄₋₂₄₀. In some cases, the exogenous VGF or peptide thereof is expressed at a higher level in the subject relative to the subject prior to delivery of the nucleic acid molecule. In some cases, the exogenous VGF or peptide thereof comprises at least 50% amino acid sequence homology to a native VGF or a peptide thereof. In some cases, the exogenous VGF or peptide thereof comprises at least 50% nucleic acid sequence homology to a native VGF or a peptide thereof. In some cases, the native VGF is derived from a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts full-length human and mouse VGF sequences.

FIG. 2 depicts human and mouse TLQP-21 and TLQP-62 peptide sequences.

FIGS. 3A-3I demonstrates that running prolongs lifespan and ameliorates motor function of mice with cerebellar ataxia. FIG. 3A depicts a Kaplan-Maier curve for Snf2h cKO (Snf2h cKO or cKO hereon) mice in sedentary (cKO-Sedentary; dotted line) or running conditions (cKO-Runner) with wheels introduced at P21 and never removed (solid line); or wheels removed at P100 (cKO-Runner, wheels out; striped line). Analysis was terminated at 1 year of age (P365). n=6-10 mice per condition. FIG. 3B depicts total body weight of cKO-Runner or Wild Type-Runner littermates (WT-Runner hereon) with wheels introduced at P25. Error bars represent ±SEM. FIG. 3C depicts total kilometers traveled for 20 days after wheels were introduced at P25. n=6, males only. FIG. 3D depicts rotarod analysis for WT-Sedentary, cKO-Sedentary, and cKO-Runner mice for 2 consecutive days 15 days post-running (wheels in at P25). *P<0.05, n=6-8 mice per condition. FIGS. 3E-G depict an open field assay for WT-Sedentary, cKO-Sedentary, and cKO-Runner mice 15 days post-running (wheels in at P25). Time in all corners, total distance traveled and velocity was averaged from 6-8 mice per genotype. *P<0.05, **P<0.01. FIGS. 3H-I depict elevated plus maze at P125 from WT-Sedentary, cKO-Sedentary, cKO-Runner, and cKO-Runner-wheels out mice (wheels in at P21-P25, wheels out at P75). *P<0.05, **P<0.01, n=6-8 mice per condition.

FIG. 4 demonstrates that voluntary running triggers neurogenesis in the hippocampus of wild type mice.

FIGS. 5A-5F demonstrates that running triggers the expansion of oligodendrocyte precursors in the ataxic hindbrain. FIGS. 5A, B depict triple immunolabeling with BrdU, NG2 and lbal through the deep cerebellar nuclei and inferior olivary nucleus of WT-Sedentary, cKO-Runner, and cKO-Sedentary mice when wheels were introduced at P21. BrdU was administered in the drinking water from P21 to P35, and sagittal brain sections analyzed at P90. Boxes highlight BrdU−, Ibal+ microglial cells, while arrows denote BrdU+, Ng2+ oligodendrocyte precursors (OPs). Note a robust increase in OPs in the inferior olivary nucleus of cKO-Runner mice. n=4 mice per condition, scale bars, 50 μm. FIGS. 5C-F depict total cell counts from 1 mm²×100 um³ confocal Z-stacks through the inferior olivary nucleus of WT-Sedentary, cKO-Sedentary, and cKO-Runner mice treated with BrdU between P21-P35 (wheels in at P21) and analyzed 55 days post-BrdU removal (P90); or 145 days post-BrdU removal (P180). Error bars represent ±SEM. *P<0.05, **P<0.01, n=4 mice per condition.

FIGS. 6A-6D demonstrates that running triggers de novo myelination and enhanced Purkinje cell arborization through the ataxic cerebellum. FIG. 6A depicts toluidine blue staining through the cerebellar vermis (molecular layer) and the deep cerebellar nuclei (white matter) of WT-Sedentary and cKO-Sedentary mice at P25, and WT-Runner and cKO-Runner mice at P150 (wheels in at P25). Note the massive appearance of myelin rings through the cKO-Runner cerebellum (arrows). PC, Purkinje cell, n=4 mice per genotype, scale bars, 104 mμ. FIG. 6B depicts transmission electron microscopy (TEM) analysis through axons within the molecular layer of WT and cKO-Sedentary mice at P25, and WT and cKO-Runner mice at P150 (wheels in at P25). Note the robust de novo myelination through axonal processes in cKO-R cerebella. Ax, axon. n=4 mice per genotype, scale bars, 2 μm. FIG. 6C depicts triple immunolabeling through the molecular layer of WT-Sedentary, cKO-Sedentary, WT-Runner, and cKO-Runner at P40 when wheels were introduced at P25 with the excitatory synaptic markers VGlut1, VGlut2, and Calbindin. DAPI stains all nuclei. Note the increased arborization of PC dendritic trees in both WT-R and cKO-R cerebella. n=4 per genotype, scale bars, 20pm. FIG. 6D depicts quantitation of the molecular layer thickness of WT-Sedentary, cKO-Sedentary, WT-Runner, and cKO-Runner at P40 mice when wheels were introduced at P25. Error bars represent ±SEM. *P<0.05, **P<0.01, n.s.=not significant, n=4 per condition.

FIGS. 7A-7B demonstrates that voluntary running triggers de novo myelination in the cerebellar vermis of ataxic mice. FIG. 7A depicts toluidine blue staining through the cerebellar vermis of WT-Sedentary and cKO-Sedentary mice at P25 (left panels), and WT-Runner and cKO-Runner mice at P150 (wheels in at P25; right panels). ML=molecular layer, PC=Purkinje cell, Ax=axon, My=myelin, n=4 mice per genotype, scale bar, 100 μm. FIG. 7B depicts transmission electron microscopy (TEM) analysis through axons within the cerebellar vermis of WT-Sedentary and cKO-Sedentary mice at P25, and WT-Runner and cKO-Runner mice at P150 (wheels introduced at P25). Note the robust de novo myelination through axonal processes in cKO-R cerebella (arrows). PC, Purkinje cell, n=4 mice per genotype, scale bars, 2 μm, except for bottom rightmost panel where scale bar, 500 nm.

FIG. 8 demonstrates that voluntary running upregulates serotonin transporter synthesis in ataxic Purkinje neurons.

FIGS. 9A-9Cdemonstrates that running upregulates synaptic transmission and growth factor synthesis in the ataxic cerebellum and brain stem. A four-way comparative analysis was performed between samples sequenced from WT-Sedentary, WT-Runner, cKO-Sedentary and cKO-Runner cerebella, identifying 2290 upregulated and 1321 downregulated genes in the cKO-R vs. WT-R analysis (FIG. 9A). Gene Ontology (GO) analyses were consistent with the findings, namely that genes involved in activity-dependent synaptic transmission were increased (FIG. 9A, B). In addition, an increase in growth factors and the exercise-induced neuropeptide precursor VGF (non-acronymic; also known as nerve-growth factor inducible) was observed (FIG. 9A). The results were then validated both as significantly upregulated in cKO-Runner vs. cKO-Sedentary cerebellum and brain stem tissue with TaqMan-based quantitative PCR (FIG. 9C).

FIGS. 10A-10H demonstrate that VGF TLQP-21 stimulates OP expansion and differentiation, while full-length VGF overexpression prolongs lifespan and triggers de novo myelination in the ataxic cerebellum. FIG. 10A shows, in a parallel experiment, cells were further supplemented with BrdU (20 nM) 6 hrs after peptide or DMSO treatment and BrdU+, NG2+ or total NG2+ cells counted in 450×450 μm² bins 42-hrs post-BrdU treatment. FIG. 10B shows that at DIV3 post-purification, cells were triple immunolabeled with NG2, a marker of OPs; MAG, a marker of differentiating OLs; and MBP, a marker of myelin-associated glycoprotein. DAPI stains all nuclei. Note the enlarged processes in OLs treated with VGF TLQP-21 vs. DMSO controls. Scale bar, 10 μm. FIG. 10C depicts quantitation of the average dendritic length 48 hrs post-peptide treatment. **P<0.01, n=4 independent coverslips per condition. FIG. 10D shows that Early region 1 (E1)/E3-deleted adenoviral vectors driving an empty cassette (Ad-Control) or full-length mouse VGF (Ad-VGF) under the regulation of the human CMV promoter were delivered via tail-injection at ˜P21 in cKO-Sedentary mice. Kaplan-Maier curves highlight the extended lifespan of cKO-Sedentary mice treated with Ad-VGF viral particles (solid line) relative to cKO-Sedentary mice treated with Ad-Empty control (dotted line). Experiments were terminated at P150 for tissue analysis. FIG. 10E depicts Taq-Man probe-based qPCR expression analysis for BDNF and VGF in selected tissues 30 days after viral delivery (˜P51). L32 was used as internal control. *P<0.05, **P<0.01, n.s.=not significant, n=4 per genotype. FIGS. 10F-G depicts TEM analysis through the molecular layer of the cerebellum (FIG. 10F) or the inferior olive (FIG. 10G) of WT-Sedentary and cKO-Sedentary mice at P60 treated with empty Ad-CT or Ad-VGF vectors at ˜P21. Note the robust myelination in the cerebellum of Ad-VGF treated cKO mice. PC=Purkinje cell; Ax=axon; My=myelin; n=6 mice per genotype, scale bars, 10 μm. FIG. 10H depicts g-ratios of axons within the molecular layer of the cerebellum and the inferior olive of WT-Sedentary and cKO-Sedentary mice at P60 treated with empty Ad-CT or Ad-VGF vectors at ˜P21. **<0.01, ˜50 axons were scored from 6 independent mice per genotype.

FIG. 11 demonstrates that the granin family proteins Chromogranin-B and VGF are robustly expressed in developing wild type OPs.

FIG. 12 depicts whole mount images from mouse hearts.

FIG. 13 depicts coronal sections from mouse hearts.

FIG. 14 depicts Western blotting from plasma of mice.

### DETAILED DESCRIPTION OF THE INVENTION

The disclosure herein provides compositions and methods for treating disease in a subject. In one aspect, compositions and methods are provided for treating neurodegenerative diseases. In another aspect, compositions and methods are provided for treating cardiovascular diseases. The compositions and methods described herein provide for delivering a therapeutic agent to a subject to treat or alleviate the symptoms of a disease. In particular, the compositions and methods provide for delivering a protein or peptide, or a nucleic acid molecule encoding a protein or peptide, to a subject to treat disease. In some aspects, the compositions and methods herein provide for the diagnosis of a subject with neurodegenerative disease.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M.J. MacPherson, B.D. Hames and G.R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

The terms “polynucleotide”, “nucleotide”, “nucleotide sequence”, and “nucleic acid” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “peptide” and “protein” are used interchangeably herein to refer to polymers of amino acids of any length. A polypeptide can be any protein, peptide, protein fragment or component thereof. A polypeptide can be a protein naturally occurring in nature or a protein that is ordinarily not found in nature. A polypeptide can consist largely of the standard twenty protein-building amino acids or it can be modified to incorporate non-standard amino acids. A polypeptide can be modified, typically by the host cell, by e.g., adding any number of biochemical functional groups, including phosphorylation, acetylation, acylation, formylati on, alkylation, methylation, lipid addition (e.g. pal mitoyl ati on, myristoylation, prenylation, etc) and carbohydrate addition (e.g. N-linked and O-linked glycosylation, etc). Polypeptides can undergo structural changes in the host cell such as the formation of disulfide bridges or proteolytic cleavage.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “biopharmaceutical” as used herein refers to any composition that includes a biologic or biologic medical product that can be utilized as a medicine or therapeutic. The biologic can be any biologic that can be used as a therapeutic agent. A biologic can be any medicinal agent that is manufactured in, extracted from, or semisynthesized from a biological source. Biologics can include, without limitation, proteins, nucleic acid molecules, cells, tissues, vaccines, blood or blood components, allergenics, gene therapies, recombinant proteins and recombinant nucleic acid molecules. A biopharmaceutical may include additional agents including, without limitation, additional therapies (biologic or synthetic chemical agents), excipients, and the like.

The terms “subject” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

Methods

In one aspect, a method is provided for treating neurodegenerative disease in a subject. In some cases, the method involves delivering to the subject a biopharmaceutical composition including a therapeutic agent. In some cases, the biopharmaceutical composition includes a nucleic acid molecule encoding a therapeutic agent. In other cases, the biopharmaceutical composition includes a therapeutic agent that can be directly administered to the subject. In some cases, the therapeutic agent is exogenous. “Exogenous” as used herein refers to any protein or peptide, or nucleic acid molecule that is derived or originates from outside of the subject. Any protein or peptide, or nucleic acid molecule encoding a protein or peptide that is delivered or administered to a subject may be referred to herein as exogenous. “Endogenous” as used herein refers to any protein or peptide, or nucleic acid molecule that originates from within the subject. In particular examples, the therapeutic agent is exogenous VGF or a peptide thereof. In some cases, a nucleic acid molecule encoding the exogenous VGF or a peptide thereof is administered to the subject suffering from neurodegenerative disease. In this example, the exogenous VGF or peptide thereof may be expressed (i.e., as a protein or peptide) in the subject. In other cases, exogenous VGF or a peptide thereof is directly administered to the subject suffering from neurodegenerative disease. The neurodegenerative disease can be, without limitation, cerebellar ataxia, multiple sclerosis (MS), amyotrophic lateral sclerosis (ALS), Friedreich's ataxia or Alzheimer's disease. In some cases, the neurodegenerative disease is a demyelinating disease such as multiple sclerosis. In some examples, the exogenous VGF or peptide thereof may induce remyelination, de novo myelination or hypermyelination in the subject. The term “remyelination” as used herein refers to the generation of new myelin sheaths on demyelinated axons of a neuron (e.g., a demyelinating disease). In some cases, remyelination may involve the propagation of new oligodendrocytes or oligodendrocyte precursor cells and/or may involve stimulating oligodendrocytes or oligodendrocyte precursor cells to produce new myelin sheaths. The term “de novo myelination” as used herein refers to the generation of new myelin sheaths at sites with no prior myelination. The term “hypermyelination” as used herein refers to the generation of or the overproduction of myelin sheaths at sites with prior myelination.

In other aspects, methods are provided for inducing remyelination, de novo myelination or hypermyelination in a subject. In some cases, the subject is suffering from a neurodegenerative disease. In some cases, the neurodegenerative disease is a demyelinating disease. In some instances, the demyelinating disease is diagnosed by identifying at least one brain lesion in the subject by magnetic resonance imaging (MRI). In some cases, the demyelinating disease is multiple sclerosis, amyotrophic lateral sclerosis (ALS), cerebellar ataxia, Friedreich's ataxia or Alzheimer's disease. In some cases, a nucleic acid molecule encoding an exogenous VGF or a peptide thereof is administered to the subject. In this example, the exogenous VGF or peptide thereof may be expressed in the subject. In other cases, a VGF protein or peptide thereof is administered to the subject. The administering may induce remyelination, de novo myelination or hypermyelination in the subject, thereby treating the demyelinating disease. In some instances, the subject has fewer brain lesions as determined by MRI after administering the therapeutic agent.

In other aspects, methods are provided for treating a cardiovascular disease in a subject. The method involves delivering to the subject a therapeutic agent of the disclosure. In some cases, a nucleic acid molecule encoding the therapeutic agent of the disclosure is administered to the subject. Additionally or alternatively, the therapeutic agent is directly administered to the subject. In particular examples, the therapeutic agent is exogenous VGF or a peptide thereof In some cases, a nucleic acid molecule encoding the exogenous VGF or a peptide thereof is administered to the subject suffering from cardiovascular disease. In this example, the exogenous VGF or peptide thereof may be expressed (i.e., as a protein or peptide) in the subject. In other cases, exogenous VGF or a peptide thereof is directly administered to the subject suffering from cardiovascular disease. The cardiovascular disease can be, without limitation, rheumatic heart disease, valvular heart disease, aneurysm, atherosclerosis, hypertension, peripheral arterial disease, angina, coronary artery disease, coronary heart disease, myocardial infarction, stroke, cerebral vascular disease, transient ischemic attacks, cardiomyopathy, pericardial disease, congenital heart disease, heart failure, atrial fibrillation, endocarditis, aortic aneurysm, renal artery stenosis, myocarditis, and cardiomegaly.

Therapeutic Agents

A therapeutic agent of the disclosure can be any molecule (e.g., protein, RNA) that is delivered to a subject. In some cases, the subject can be a patient suffering from a disease or a condition. The therapeutic agent can be used to treat a disease or condition or can be used to alleviate the symptoms of a disease or condition. In some cases, the subject is healthy and the therapeutic agent is used as a prophylactic treatment to prevent the onset of a disease or condition. In other cases, the therapeutic agent is delivered to elicit a desired beneficial response in the subject. In one non-limiting example, the therapeutic agent may improve the brain function of an otherwise healthy subject.

VGF

VGF (non-acronymic) or VGF nerve growth factor inducible is a neuropeptide precursor and growth factor that is expressed in the nervous system. VGF undergoes endoproteolytic cleavage to produce one or more bioactive VGF-derived peptides. Several VGF-derived peptides have been identified, which are named by the first 4 amino acids and their overall length.

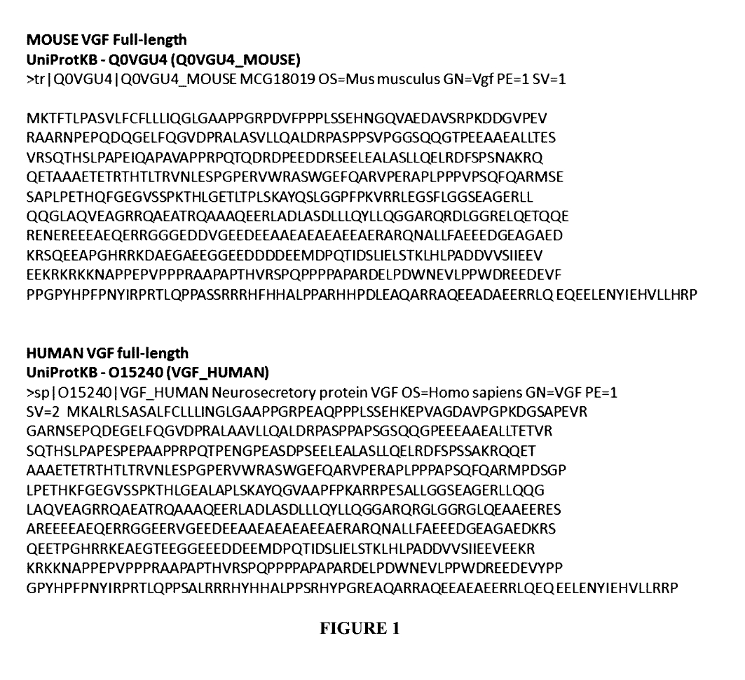

In some aspects, a therapeutic agent as envisioned herein includes VGF and any VGF-derived peptide or fragment thereof. In some cases, the therapeutic agent is full-length VGF. Full-length VGF may refer to the precursor VGF that has not undergone any proteolytic cleavage. Non-limiting examples of human and mouse full-length VGF sequences are depicted in FIG. 1. Full-length VGF may elicit a therapeutic effect directly in the subject or may undergo further processing (i.e., proteolytic cleavage) to generate bioactive VGF-derived peptides or fragments. It should be understood that full-length VGF can refer to any VGF protein that is substantially full-length or may be less than a full-length as compared to a native VGF sequence. The term “native” as used herein refers to that which is ordinarily found in nature. For example, a “native” VGF may be any VGF protein that is naturally occurring. The term “native” may refer to the protein itself, or to the amino acid and/or nucleic acid sequences. A full-length VGF can refer to a VGF protein that includes at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% of the size of a full-length native VGF. Non-limiting examples of full-length native VGF amino acid sequences are depicted in FIG. 1.

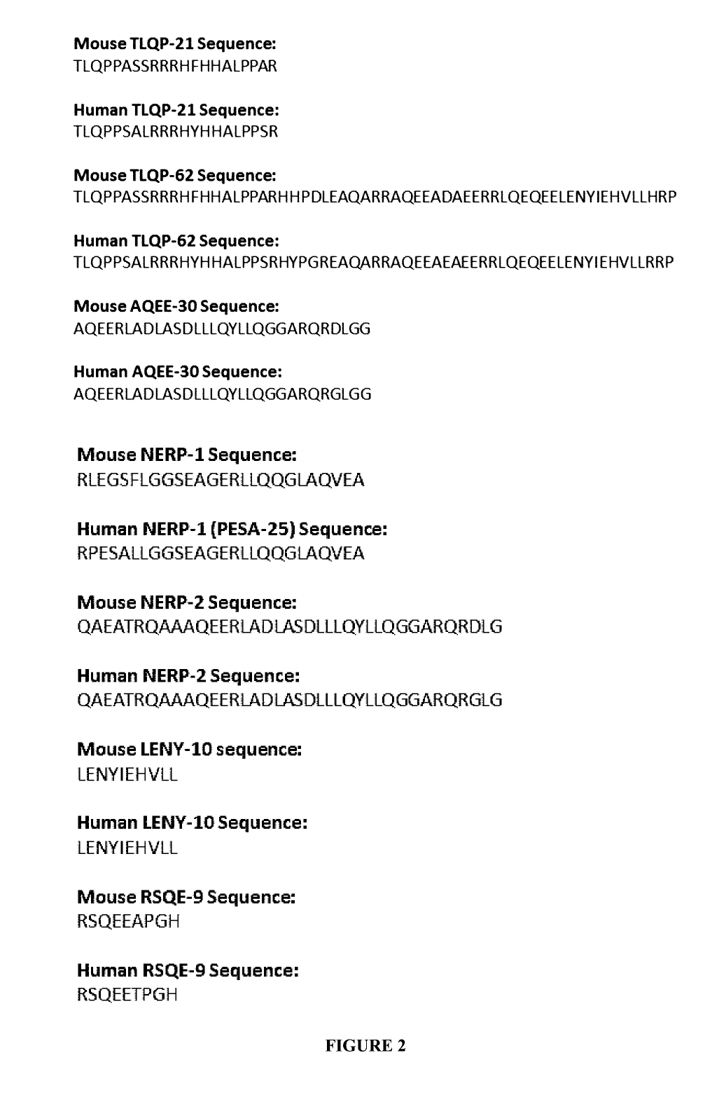

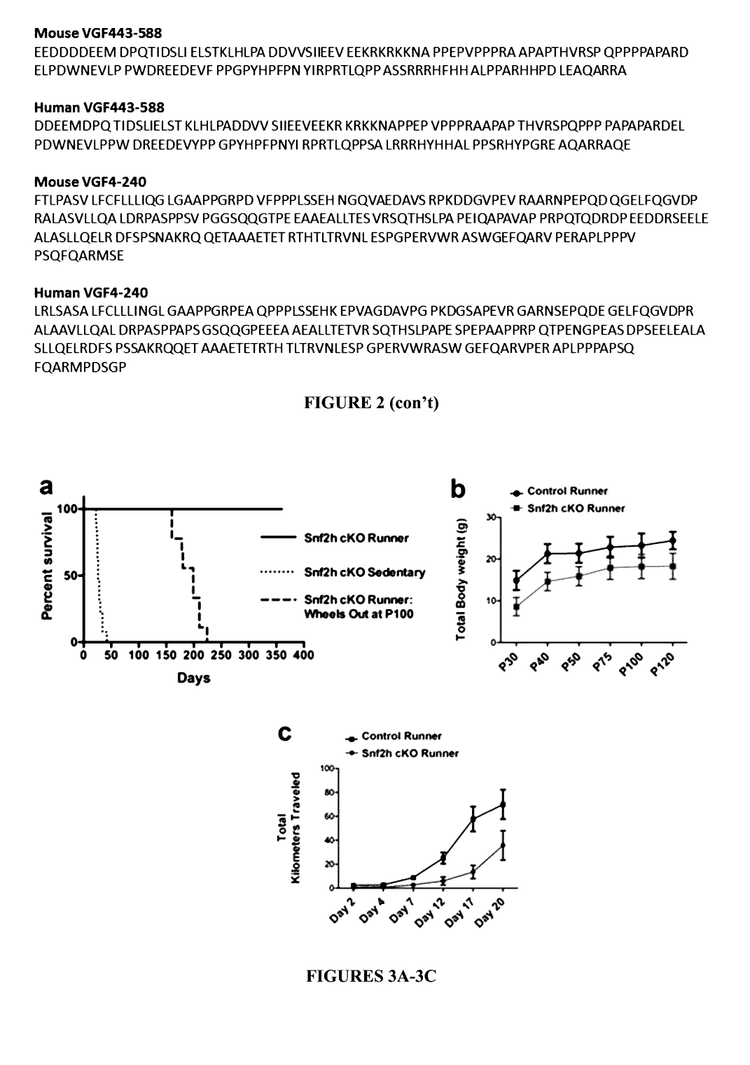

In some cases, the therapeutic agent is a VGF-derived peptide or fragment. In some cases, the VGF-derived peptide is TLQP-21, TLQP-62, AQEE-30, NERP-1, NERP-2, LENY-10, RSQE-9, VGF₄₄₃₋₅₈₈, and VGF₄₋₂₄₀, the sequences of which are depicted in FIG. 2. In a particular example, the therapeutic agent is TLQP-21.

VGF or a peptide thereof may be derived from any species. In some cases, VGF or a peptide thereof is derived from a mammal including, without limitation, human, mouse, rat, rabbit, dog, cat, cow, sheep, donkey, horse, pig, guinea pig, and non-human primates (e.g., chimpanzee, macaque, etc.). In a particular example, VGF or a peptide thereof is derived from human.

VGF or a peptide thereof may include the native VGF amino acid sequence or can be substantially similar to the native VGF amino acid sequence. For example, a full-length VGF may include at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% amino acid sequence homology or identity to a native full-length VGF. In some cases, a VGF peptide may include at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% amino acid sequence homology or identity to a native VGF peptide.

VGF or a peptide thereof may include the native VGF nucleic acid sequence or can be substantially similar to the native VGF nucleic acid sequence. For example, a full-length VGF may include at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% nucleic acid sequence homology or identity to a native full-length VGF. In some cases, a VGF peptide may include at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% nucleic acid sequence homology or identity to a native VGF peptide.

In some cases, VGF or a VGF-derived peptide can be administered or delivered exogenously to a subject as a protein or peptide or can be administered or delivered by a nucleic acid molecule (e.g., in a vector) encoding the exogenous VGF or VGF-derived peptide. It should be understood that a subject may express endogenous VGF or VGF-derived peptide prior to and/or after delivery of the exogenous vector or protein. In some cases, administration of VGF or a VGF-derived peptide to a subject may increase the total VGF or VGF-derived peptide levels produced or present in the subject as compared to the subject e.g., prior to administration. Additionally or alternatively, administration of exogenous VGF or VGF-derived peptide to a subject may replenish the VGF or VGF-derived peptide levels in a subject that no longer produces VGF (e.g., due to pathology). In some cases, exogenous VGF or VGF-derived peptide induces remyelination in a subject. In other cases, exogenous VGF or VGF-derived peptide induces de novo myelination in a subject. In some cases, exogenous VGF or VGF-derived peptide induces hypermyelination in a subject.

In some cases, the therapeutic agent may be a protein that induces the expression of endogenous VGF in a subject. Non-limiting examples of molecules that can induce the expression of endogenous VGF (i.e., an upstream effector) include: members of the brain-derived neurotrophic factor (BDNF)/tropomyosin receptor kinase B (TrkB)/cAMP response element-binding protein (CREB) signaling pathway, members of the phosphatidylinositol 3-kinase (PI3K)/protein kinase B (AKT)/mammalian target of rapamycin (mTOR) signaling pathway, nerve growth factor (NGF), glial cell-line derived neurotrophic factor (GDNF), RET oncoproteins, EP300, neuron-restrictive silencer factor (NRSF), and chromogranin-B (ChgB). An upstream effector of VGF can be administered as a therapeutic agent by any method described herein to a subject to treat neurodegenerative or cardiovascular disease. Additionally or alternatively, the therapeutic agent may be a downstream effector of VGF. A downstream effector of VGF can be any protein upregulated and/or activated by VGF (e.g., a transcription factor, a signaling protein, a receptor, etc.). A downstream effector of VGF can be administered as a therapeutic agent by any method described herein to a subject to treat neurodegenerative or cardiovascular disease. Non-limiting examples of downstream effectors of VGF include Atp1b2, Cuzd1, Dgcr2, Fat1, Fat4, Lpp, Ptk7, Tmem8b, 2610005L07Rik, Vwf, Adam12, Aoc3, App, Antxr1, Astn1, Bmpr1b, Abl1, Cdh13, Celsr2, Ctnnd1, Ctnnd2, Chl1, Cldn15, Col11a1, Col12a1, Cntn3, Cyfip2, Dchs1, Ddr1, Dst, Fndc3a, HapIn4, Inppl1, Itga4, Itga6, Itga8, Itgav, Itgb8, Lama5, Lamc1, Lsamp, Neo1, Ncam2, Nrxn1, Ncan, Nlgn2, Nid2, Pard3, Parva, Pxn, Hspg2, Pvr, Pvr13, Pkd1, Ptprf, Pcdh1, Pcdh10, Pcdh11x, Pcdh17, Pcdh7, Pcdha3, Pcdha8, Pcdhga1, Pcdhga10, Pcdhga4, Pcdhga5, Pcdhga6, Pcdhga7, Pcdhgal1, Pcdhgb4, Pcdhgc5, Pcdhgc4, Pcdhga3, Pcdhga12, Pcdhga9, Pcdhga3, Pcdhga8, Pcdhga2, Pcdhgb1, Pcdhgb2, Pcdhgb5, Pcdhgb6, Pcdhgb7, Pcdhgb8, Scarb2, Scrib, Siglec1, Sdk1, Sd1c2, Pcdha9, Pcdha6, Pcdha4, Pcdha12, Pcdha1, Pcdha5, Pcdha11, Pcdha10, Pcdhac2, Pcdha7, Pcdhac1, Pcdha2, Sorbs1, Spon1, Sned1, Sympk, Tnr, Trpm7, Tnfrsfl2a, Ttyh1, Ab12, Vc1, Dhcr24, Hmgcr, Dhcr7, Nsdh1, 0610007P14Rik, Cyp51, Insig1, Idi1, Mvd, Myk, Pmvk, Fdft1, Prkaa2, Fdps, Sc4mol, Sc5d, and Tm7s12. In a particular example, the therapeutic agent is an integrin (e.g., integrin alpha 4 (Itga4), integrin alpha 6 (Itga6), integrin alpha 8 (Itga8), integrin alpha V (ItgaV), and integrin beta 8 (Itgb8)). In other particular examples, the therapeutic agent is a protocadherin isoform. In some cases, the therapeutic agent is a receptor that recognizes and/or is activated by VGF or a VGF-derived peptide. For example, in some cases, the therapeutic agent is gClqR (the globular heads of the Clq receptor). In another example, the therapeutic agent is C3AR1 (complement C3a receptor-1). In some cases, the VGF or VGF-derived peptide receptor may be constitutively active (i.e., activated in the absence of an agonist).

Delivery Methods

In some cases, therapeutic agents of the disclosure can include a nucleic acid molecule encoding a protein or a peptide. The term “nucleic acid” as used herein generally refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecules can be DNA or RNA, or any combination thereof. RNA can comprise mRNA, miRNA, piRNA, siRNA, tRNA, rRNA, sncRNA, snoRNA and the like. DNA can comprise cDNA, genomic DNA, mitochondrial DNA, exosomal DNA, viral DNA and the like.

In some cases, nucleic acid molecules are delivered to a subject substantially free of other agents (i.e., “naked” DNA or RNA). In other cases, the nucleic acid molecule is delivered to a subject in the form of a vector. The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. A vector can deliver a nucleic acid molecule of interest to an organism, a cell or a cellular component. In some cases, the vector is an expression vector. An “expression vector” as used herein refers to a vector, for example, a plasmid, that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In particular cases, a vector or expression vector is used to deliver a nucleic acid molecule encoding a therapeutic agent of the disclosure to a subject. A nucleic acid sequence encoding any therapeutic agent of the disclosure may be referred to herein as the “target gene.” The target gene may include a coding sequence and any regulatory elements that control the expression of the gene. The therapeutic agent may be referred to herein as the “target agent”, “target protein”, or “target gene product”. Therapeutic agents encoded by a nucleic acid molecule will generally be proteins or peptides but can also encompass any molecule encoded by the nucleic acid sequence, such as small RNAs (e.g., miRNA, siRNA, and the like).

In some cases, the vector is a circular nucleic acid, for e.g., a plasmid, a BAC, a PAC, a YAC, a cosmid, a fosmid, and the like. In some cases, circular nucleic acid molecules can be utilized to deliver a nucleic acid molecule encoding a therapeutic agent to a subject. For example, a plasmid DNA molecule encoding a therapeutic agent can be introduced into a cell of a subject whereby the DNA sequence encoding the therapeutic agent is transcribed into mRNA and the mRNA “message” is translated into a protein product. The circular nucleic acid vector will generally include regulatory elements that regulate the expression of the target protein. For example, the circular nucleic acid vector may include any number of promoters, enhancers, terminators, splice signals, origins of replication, initiation signals, and the like. In some cases, the backbone of the vector may be derived from an organism other than that of the target gene. For instance, the backbone of a plasmid may be derived from a bacterial source whereas the

In some cases, the nucleic acid molecule can include a replicon. A replicon may be any nucleic acid molecule capable of self-replication. In some cases, the replicon is an RNA replicon based on or derived from viruses. A variety of suitable viruses (e.g. RNA viruses) are available, including, but not limited to, alphavirus, picornavirus, flavivirus, coronavirus, pestivirus, rubivirus, calcivirus, and hepacivirus.

In some cases, the vector is a viral vector. In some cases, the viral vector is based on or derived from a replication-deficient virus. Non-limiting examples of viral vectors suitable for delivering a nucleic acid molecule of the disclosure to a subject include those derived from adenovirus, retrovirus (e.g., lentivirus), adeno-associated virus (AAV), and herpes simplex-1 (HSV-1). In a particular case, the viral vector is derived from AAV and may be any AAV serotype. In some cases, the viral vector may be derived from AAV-1, AAV-2, AAV-3, AAV-4, AAV-5, AAV-7, AAV-8, or AAV-9. In particular cases, the viral vector is derived from AAV-2 or A AV9.

In some cases, the vector may deliver a nucleic acid sequence for targeted expression in a subject. “Targeted expression” or “selective expression” are used interchangeably herein and refer to the directed or specific expression of a protein or peptide (i.e., therapeutic agent) in a specific tissue or cell-type of the subject. Targeted expression of the protein or peptide may involve any number of promoters or regulatory elements. In a particular example, a therapeutic agent of the disclosure is selectively expressed in glial cells. In some cases, the glial cells are oligodendrocytes or oligodendrocyte precursor cells. In some cases, a vector may comprise a neural/glial antigen 2 (NG2) promoter, a platelet-derived growth factor (PDGF)-alpha promoter, a myelin basic protein (MBP) promoter, a VGF promoter, a myelin oligodendrocyte glycoprotein (MOO) promoter, a myelin-associated glycoprotein (MAG) promoter, an oligodendrocyte transcription factor 1 (Olig1), an oligodendrocyte transcription factor 2 (Olig2) promoter, or an OP4 promoter to target expression of the therapeutic agent to oligodendrocytes or oligodendrocyte precursor cells.

In another embodiment, the invention includes inducing expression at the endogenous VGF locus in the genome of a cell to be treated. Technologies for engineering synthetic transcription factors have enabled many advances in medical and scientific research. In contrast to existing methods based on engineering of DNA-binding proteins, one of skill in the art can create a Cas9-based transactivator, for example, that is targeted to DNA sequences by guide RNA molecules. Coexpression of this transactivator and combinations of guide RNAs in human cells induce specific expression of endogenous target genes, such as VGF. By way of example, structure-guided engineering of a CRISPR-Cas9 complex to mediate efficient transcriptional activation at endogenous genomic loci for VGF can be designed.

Indications

In some aspects, the compositions and methods disclosed herein provide for treating a neurodegenerative disease in a subject. Non-limiting examples of neurodegenerative diseases include Alzheimer's disease, Senile dementia of the Alzheimer type, or Pick's disease (lobar atrophy), syndromes combining progressive dementia with other prominent neurologic abnormalities, Huntington's disease, multiple system atrophy combining dementia with ataxia and/or manifestation of Parkinson's disease, progressive supranuclear palsy (Steele-Richardson-Olszewski), diffuse Lewy body disease, or corticodentatonigral degeneration, Hallervorden-Spatz disease and progressive familial myoclonic epilepsy, symptoms of gradually developing abnormalities of posture and movement, paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, or Gilles de la Tourette syndrome, progressive ataxia, cerebellar degenerations or spinocerebellar degenerations, cerebellar cortical degeneration or olivopontocerebellar atrophy (OPCA), spinocerebellar degenerations (Friedreich's ataxia and related disorders), central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), or other forms of familial spinal muscular atrophy, primary lateral sclerosis, or hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, syndromes of progressive visual loss, pigmentary degeneration of the retina (retinitis pigmentosa), or hereditary optic atrophy (Leber's disease), Parkinson's disease and other extrapyramidal disorders, progressive supranuclear palsy (Steele-Richardson-Olszewski syndrome), torsion dystonia (torsion spasm, dystonia musculorum deformans), focal dystonias, Familial tremors, or Gilles de la Tourette syndrome, motor neuron disease and the progressive ataxias, amyotrophic lateral sclerosis, primary lateral sclerosis, multifocal motor neuropathy with conduction block, motor neuropathy with paraproteinemia, motor-predominant peripheral neuropathies, olivopontocerebellar atrophy, Azorean (Machado-Joseph) disease, familial progressive neurodegenerative diseases, familial amyotrophic lateral sclerosis, spinal muscular atrophies, familial spastic paraparesis, hereditary biochemical disorders, arthrogryposis multiplex congenital, or progressive juvenile bulbar palsy (Fazio-Londe), infantile (Werdnig-Hoffman disease), childhood onset, or adolescent (Wohlfart-Kugelberg-Welander disease), familial HTLV-1 myelopathy, isolated FSP, or complicated FSP, superoxide dismutase deficiency, hexosaminidase A and B deficiency, or androgen receptor mutation (Kennedy's syndrome), viral and prion diseases, myelopathy, progressive multifocal leukoencephalopathy, Creutzfeldt-Jakob disease, Gerstmann-Straussler-Scheinker disease, kuru, fatal familial insomnia, or Alper's disease, includes primary progressive or secondary progressive multiple sclerosis, fronlolempural dementia, Wilson's disease, progressive neuropathic pain, and spinocerebellar ataxias (SCM, SCA2, SCA3, SCA4, SCA5, SCA6, SCA7, SCA8, SCA10, SCA11, SCA12, SCA13, SCA14, SCA15, SCA16, SCA17, SCA21, SCA22, SCA23, and SCA25). In a particular example, the neurodegenerative disease is multiple sclerosis, amyotrophic lateral sclerosis, Friedreich's ataxia, spinocerebellar ataxia (all types) or Alzheimer's disease.

In some cases, the neurodegenerative disease is a demyelinating disease. In this example, the therapeutic agent of the disclosure may induce remyelination, de novo myelination or hypermyelination in a subject suffering from demyelinating disease. Non-limiting examples of demyelinating disease include multiple sclerosis, neuromyelitis optica, optic-spinal multiple sclerosis, chronic relapsing inflammatory optic neuritis, acute disseminated encephalomyelitis, acute hemorrhagic leukoencephalitis, balo concentric sclerosis, Schilder disease, Marburg multiple sclerosis, tumefactive multiple sclerosis, solitary sclerosis, Susac's disease, leukoaraiosis, myalgic encephalomyelitis, Guillain-Barr syndrome, progressive inflammatory neuropathy, leukodystrophy, adrenoleukodystrophy, adrenomyeloneuropathy, venous induced demyelination, primary progressive multiple sclerosis, aquaporine-related multiple sclerosis, clinically isolated syndrome, central pontine myelinolysis, Alexander disease, Canavan disease, Krabbe disease, metachromatic leukodystrophy, Pelizaeus-Merzbacher disease, leukoencephalopathy with vanishing white matter, megalencephalic leukoencephalopathy with subcortical cysts, CAMFAK syndrome, Marchiafava-Bignami disease, Alper's disease, transverse myelitis, syphilitic myelopathy, progressive multifocal leukoencephalopathy, toxic leukoencephalopathy, leukoencephalopathy with neuroaxonal spheroids, posterior reversible encephalopathy syndrome, hypertensive leukoencephalopathy, Refsum disease, xenobefantosis, chronic inflammatory demyelinating polyneuropathy, Charcot-Marie-Tooth disease, copper deficiency disorders, and progressive inflammatory neuropathy.

In some cases, the compositions and methods are provided herein for treating cardiovascular disease. Non-limiting examples of cardiovascular disease include rheumatic heart disease, valvular heart disease, aneurysm, atherosclerosis, hypertension, peripheral arterial disease, angina, coronary artery disease, coronary heart disease, myocardial infarction, stroke, cerebral vascular disease, transient ischemic attacks, cardiomyopathy, pericardial disease, congenital heart disease, heart failure, atrial fibrillation, endocarditis, aortic aneurysm, renal artery stenosis, myocarditis, and cardiomegaly.

Biopharmaceutical Compositions

The disclosure herein provides for biopharmaceutical compositions that may be used to treat neurodegenerative disease. In other examples, the biopharmaceutical compositions may be used to treat cardiovascular disease. In some aspects, the composition can be a solid formulation, particularly useful for e.g., oral administration to a subject in need thereof. In some aspects, the solid formulation may include a therapeutic agent of the disclosure or a nucleic acid molecule encoding the therapeutic agent (e.g., VGF or VGF-derived peptide). In some cases, the therapeutic agent may be present in the composition at an amount, for example, of about 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 120 μg, about 140 μg, about 160 μg, about 180 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1000 mg, or greater than 1000 mg.

In some aspects, the composition can be a liquid formulation used for, e.g., injection into a subject in need thereof. In some examples, a liquid formulation may include a therapeutic agent of the disclosure or a nucleic acid molecule encoding the therapeutic agent (e.g., VGF or VGF-derived peptide) at a concentration of about 1 μg /mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 120 μg/mL, about 140 μg/mL, about 160 μg/mL, about 180 μg/mL, about 200 μg/mL, about 220 μg/mL, about 240 μg/mL, about 260 μg/mL, about 280 μg/mL, about 300 μg/mL, about 320 μg/mL, about 340 μg/mL, about 360 μg/mL, about 380 μg/mL, about 400 μg/mL, about 420 μg/mL, about 440 μg/mL, about 460 μg/mL, about 480 μg/mL, about 500 μg/mL, about 520 μg/mL, about 540 μg/mL, about 560 μg/mL, about 580 μg/mL, about 600 μg/mL, about 620 μg/mL, about 640 μg/mL, about 660 μg/mL, about 680 μg/mL, about 700 μg/mL, about 720 μg/mL, about 740 μg/mL, about 760 μg/mL, about 780 μg/mL, about 800 μg/mL, about 820 μg/mL, about 840 μg/mL, about 860 μg/mL, about 880 μg/mL, about 900 μg/mL, about 920 μg/mL, about 940 μg/mL, about 960 μg/mL, about 980 μg/mL, 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, about 200 mg/mL, about 220 mg/mL, about 240 mg/mL, about 260 mg/mL, about 280 mg/mL, about 300 mg/mL, about 320 mg/mL, about 340 mg/mL, about 360 mg/mL, about 380 mg/mL, about 400 mg/mL, about 420 mg/mL, about 440 mg/mL, about 460 mg/mL, about 480 mg/mL, about 500 mg/mL, about 520 mg/mL, about 540 mg/mL, about 560 mg/mL, about 580 mg/mL, about 600 mg/mL, about 620 mg/mL, about 640 mg/mL, about 660 mg/mL, about 680 mg/mL, about 700 mg/mL, about 720 mg/mL, about 740 mg/mL, about 760 mg/mL, about_{.} 780 mg/mL, about 800 mg/mL, about 820 mg/mL, about 840 mg/mL, about 860 mg/mL, about 880 mg/mL, about 900 mg/mL, about 920 mg/mL, about 940 mg/mL, about 960 mg/mL, about 980 mg/mL, about 1000 mg/mL, or greater than 1000 mg/mL.

Compositions as described herein may include a liquid formulation, a solid formulation, or a combination thereof. Non-limiting examples of formulations may include a tablet, a capsule, a gel, a paste, a liquid solution, cream or an aerosol (i.e., a spray). In some instances, the therapeutic agent or drug may be in a crystallized form. Solid formulations may be suitable for oral administration of the composition to a subject in need thereof. In some cases, slow release formulations for oral administration may be prepared in order to achieve a controlled release of the active agent in contact with the body fluids in the gastrointestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

The compositions of the present disclosure may further include any number of excipients. Excipients may include any and all solvents, coatings, flavorings, colorings, lubricants, disintegrants, preservatives, sweeteners, binders, diluents, and vehicles (or carriers). Generally, the excipient is compatible with the therapeutic compositions of the present disclosure.

Dosage and Administration

Suitable doses of formulations of the disclosure can be administered to a subject in need thereof. Non-limiting examples of methods of administration include subcutaneous administration, intravenous administration, intramuscular administration, intradermal administration, intraperitoneal administration, oral administration, infusion, intracranial administration, intrathecal administration, intranasal administration, and oral administration. In some cases, administration can involve injection of a formulation of the composition. In other cases, administration can involve oral delivery of a solid formulation of the composition. In particular cases, the administration is oral administration. In some cases, the oral composition can be administered with food. In other particular cases, the administration is intracranial injection. In yet other particular cases, the administration is intravenous injection. In other particular examples, the therapeutic agent is delivered to the cerebrospinal fluid (CSF) by e.g., intrathecal administration.

In some aspects, a therapeutically effective amount of the therapeutic agent is administered. A “therapeutically effective amount” or “therapeutically effective dose” is an amount of a therapeutic agent that provokes a therapeutic or desired response in a subject. A therapeutically effective amount or dose of a composition of the disclosure can be expressed as mg of the composition per kg of subject body mass. In some instances, a therapeutically effective amount may be about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about lOps/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 120 μg/kg, about 140 μg/kg, about 160 μg/kg, about 180 μg/kg, about 200 μg/kg, about 220 μg/kg, about 240 μg/kg, about 260 μg/kg, about 280 μg/kg, about 300 μg/kg, about 320 μg/kg, about 340 μg/kg, about 360 μg/kg, about 380 μg/kg, about 400 μg/kg, about 420 μg/kg, about 440 μg/kg, about 460 μg/kg, about 480 μg/kg, about 500 μg/kg, about 520 μg/kg, about 540 μg/kg, about 560 μg/kg, about 580 μg/kg, about 600 μg/kg, about 620 μg/kg, about 640 μg/kg, about 660 μg/kg, about 680 μg/kg, about 700 μg/kg, about 720 μg/kg, about 740 μg/kg, about 760 μg/kg, about 780 μg/kg, about 800 μg/kg, about 820 μg/kg, about 840 μg/kg, about 860 μg/kg, about 880 μg/kg, about 900 μg/kg, about 920 μg/kg, about 940 μg/kg, about 960 μg/kg, about 980 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, about 320 mg/kg, about 340 mg/kg, about 360 mg/kg, about 380 mg/kg, about 400 mg/kg, about 420 mg/kg, about 440 mg/kg, about 460 mg/kg, about 480 mg/kg, about 500 mg/kg, about 520 mg/kg, about 540 mg/kg, about 560 mg/kg, about 580 mg/kg, about 600 mg/kg, about 620 mg/kg, about 640 mg/kg, about 660 mg/kg, about 680 mg/kg, about 700 mg/kg, about 720 mg/kg, about 740 mg/kg, about 760 mg/kg, about 780 mg/kg, about 800 mg/kg, about 820 mg/kg, about 840 mg/kg, about 860 mg/kg, about 880 mg/kg, about 900 mg/kg, about 920 mg/kg, about 940 mg/kg, about 960 mg/kg, about 980 mg/kg, about 1000 mg/kg, or greater than 1000 mg/kg.

The composition can be administered once or more than once each day. In some cases, the composition is administered as a single dose (i.e., one-time use). In this example, the single dose may be curative. In other cases, the composition may be administered serially (e.g., taken every day without a break for the duration of the treatment regimen). In some cases, the treatment regime can be less than a week, a week, two weeks, three weeks, a month, or greater than a month. In some cases, the composition is administered over a period of at least 12 weeks. In other cases, the composition is administered for a day, at least two consecutive days, at least three consecutive days, at least four consecutive days, at least five cons'ecutive days, at least six consecutive days, at least seven consecutive days, at least eight consecutive days, at least nine consecutive days, at least ten consecutive days, or at least greater than ten consecutive days. In some cases, a therapeutically effective amount can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount can be administered one time per day, two times per day, three times per day, four times per day, five times per day, six times per day, seven times per day, eight times per day, nine times per day, 10 times per day, or greater than 10 times per day. In some cases, the composition is administered at least twice a day. In further cases, the composition is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day.

Diagnostics

In one aspect, a method is provided for diagnosing a subject with a neurodegenerative disease. In some cases, the method may involve detecting a level of VGF protein or a peptide thereof in a sample of the subject. The sample may be blood or a component thereof (i.e., serum, plasma) or cerebrospinal fluid (CSF). In a particular case, the sample is plasma. In some cases, the method involves comparing the level of VGF protein or peptide thereof to the level of VGF protein or peptide thereof in a healthy control. The subject may be diagnosed with a neurodegenerative disease if the level of VGF protein or peptide thereof is lower in the subject as compared to the level of VGF protein or peptide thereof in the healthy control. In particular cases, the subject is diagnosed with Alzheimer's disease if the level of VGF protein or peptide thereof is lower in the subject as compared to the level of VGF protein or peptide thereof in a healthy control. In other particular cases, the subject is diagnosed with multiple sclerosis if the level of VGF protein or peptide thereof is lower in the subject as compared to the level of VGF protein or peptide thereof in a healthy control. In some cases, the subject is diagnosed with a neurodegenerative disease if the level of VGF is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or greater than 100-fold lower than the level of VGF in a healthy control. In some cases, the determination of VGF levels may be combined with another diagnostic marker in order to diagnose a subject with a neurodegenerative disease. For example, a subject may be diagnosed with multiple sclerosis if the VGF levels are lower than that of a healthy control and the subject is positive for oligoclonal bands.

Additionally or alternatively, a method is provided for diagnosing a subject with cardiovascular disease. In a particular case, the method can be used to diagnose a subject with congenital heart disease. In some cases, the method may involve detecting a level of VGF protein or a peptide thereof in a sample of the subject. The sample may be blood or a component thereof (i.e., serum, plasma) or cerebrospinal fluid (CSF). In a particular case, the sample is plasma. In some cases, the method involves comparing the level of VGF protein or peptide thereof to the level of VGF protein or peptide thereof in a healthy control. In particular cases, the subject is diagnosed with congenital heart disease if the level of VGF protein or peptide thereof is lower in the subject as compared to the level of VGF protein or peptide thereof in a healthy control. The subject may be diagnosed with a cardiovascular disease if the level of VGF protein or peptide thereof is lower in the subject as compared to the level of VGF protein or peptide thereof in the healthy control. In some cases, the subject is diagnosed with a cardiovascular disease if the level of VGF is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or greater than 100-fold lower than the level of VGF in a healthy control. In some cases, the determination of VGF levels may be combined with another diagnostic marker in order to diagnose a subject with a cardiovascular disease.

In some cases, the method may involve collecting a sample from a subject and subjecting the sample to one or more assays to detect a presence or absence of one or more biomarkers. In some cases, the one or more biomarkers include VGF, VGF-derived peptide or downstream effectors thereof. The one or more biomarkers may be detected at the protein expression level or may be detected at the nucleic acid expression level. The one or more assays can be any method of detecting the presence or absence of the one or more biomarkers. In one example, the one or more assays may involve an enzyme-linked immunosorbent assay (ELISA) to detect the level of VGF protein or VGF-derived peptide present in a biological sample. The ELISA assay may involve one or more antibodies that recognize one or more VGF proteins or peptides. Additionally or alternatively, the one or more assays may involve mass spectrometry. Mass spectrometry may be utilized to detect the presence of VGF or VGF-derived peptide in a biological sample (i.e., by measuring the mass-to-charge ratio). In some examples, the one or more assays may include gene expression profiling by any number of methods known to those of skill in the art including microarray, RNAseq, reverse transcription polymerase chain reaction (RT-PCR), and the like.

In some cases, a subject diagnosed with a neurodegenerative or a cardiovascular disease by the methods described above is treated for the respective disease. In some cases, the subject is treated with a therapeutic agent of the disclosure. In a particular example, the subject is treated with VGF or a VGF-derived peptide. In this example, the VGF or VGF-derived peptide may replenish decreased VGF or VGF-derived peptide levels in the subject. In some cases, the subject is diagnosed with a demyelinating disease. In this example, the VGF or VGF-derived peptide may induce remyelination, de novo myelination or hypermyelination in the subject. In some cases, the subject is treated with a combination therapy that includes a therapeutic agent of the disclosure and an additional therapy. In some cases, the combination therapy includes VGF or a VGF-derived peptide and an additional therapy. For example, in some cases, VGF or a VGF-derived peptide is combined with an immunomodulatory therapy. Non-limiting examples of immunomodulatory therapies include Interferon beta-1a (Avonex, Rebif), Interferon beta-1b (Betaseron, Extavia), Peginterferon beta-1a (Plegridy), Glatiramer acetate (Copaxone), Natalizumab (Tysabri), Mitoxantrone, Fingolimod (Gilenya), Teriflunomide (Aubagio), Dimethyl fumarate (Tecfidera), and Alemtuzumab (Lemtrada).

In some aspects, a method is provided for monitoring a response of a subject to a treatment. In some instances, the method provides for measuring a baseline level of VGF or a downstream effector of VGF (e.g., integrin) in the subject prior to treatment. The subject may be treated with a therapeutic agent of the disclosure (e.g., VGF). The subject may be screened post-treatment for the presence or absence of VGF or VGF-derived peptide levels. In some cases, the subject may be screened for the presence or absence of downstream effectors of VGF (e.g., integrins). The levels of VGF or a downstream effector thereof in a subject post-treatment may be compared to the baseline levels of VGF or a downstream effector thereof in the same subject. In some cases, the levels of VGF or a downstream effector thereof in the subject may be compared to the levels of VGF or a downstream effector thereof in a healthy control. An increased level of VGF or a downstream effector thereof over baseline levels can indicate that the subject is responsive to the treatment, whereas an absence of or a lower level of VGF or a downstream effector thereof in the subject post-treatment as compared to a healthy control may indicate that the subject is unresponsive to the treatment.

Kits

The disclosure herein also provides for kits. Kits can include any component suitable to perform the methods of the disclosure. A kit may include a pharmaceutical composition. In particular cases, the kit may include one or more pharmaceutical compositions suitable for treating neurodegenerative disease in a subject. In other particular cases, a kit may include one or more pharmaceutical compositions for treating cardiovascular disease in a subject. A pharmaceutical composition may include a therapeutic agent of the disclosure. The therapeutic agent can be provided in one or more therapeutically effective doses. The therapeutic agent can be in any composition (i.e., formulation) as described herein.

In one case, a therapeutic agent includes a VGF protein or a peptide thereof. The VGF protein or peptide thereof can be any VGF protein or peptide (e.g., TLQP-21) and of any formulation and dosage as described herein. Additionally or alternatively, the kit can include a nucleic acid molecule encoding VGF protein or peptide and of any formulation and any dosage as described herein. In some cases, the kit may include a therapeutically effective dose of a biopharmaceutical composition in a pill or tablet formulation for oral administration to a subject. In other cases, the kit may include a therapeutically effective dose of a biopharmaceutical composition in a liquid formulation for e.g., intracranial, intramuscular, intravenous, intrathecal administration. A biopharmaceutical composition can be provided in any formulation as described herein (e.g., a tablet, a gel, a cream, and the like). In some cases, the kit may include a biopharmaceutical composition in a dried (i.e., powder) form. The dried or powdered form may be reconstituted with a liquid solution (e.g., a saline solution) to generate a liquid formulation. Kits may further include one or more excipients as described herein (e.g., a preservative, a carrier, etc.).

The kit may further include a means (e.g., a device) for delivering the biopharmaceutical composition to a subject. For example, a kit may include one or more needles suitable for injectable administration. The kit may further include one or more alcohol wipes for sterilization of the injection site.

In some cases, kits may be provided with instructions. The instructions may be provided in the kit or they may be accessed electronically (e.g., on the World Wide Web). The instructions may provide information on how to use the compositions of the present disclosure. The instructions may provide information on how to perform the methods of the disclosure. In some cases, the instructions may provide dosing information. In some cases, the instructions may provide drug information such as the mechanism of action, the formulation of the drug, adverse risks, contraindications, and the like. In some cases, the kit is purchased by a physician or health care provider for administration at a clinic or hospital. In other cases, the kit is purchased by the subject and self-administered.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Running Triggers De Novo Myelination to Prolong the Lifespan of Mice with Cerebellar Ataxia

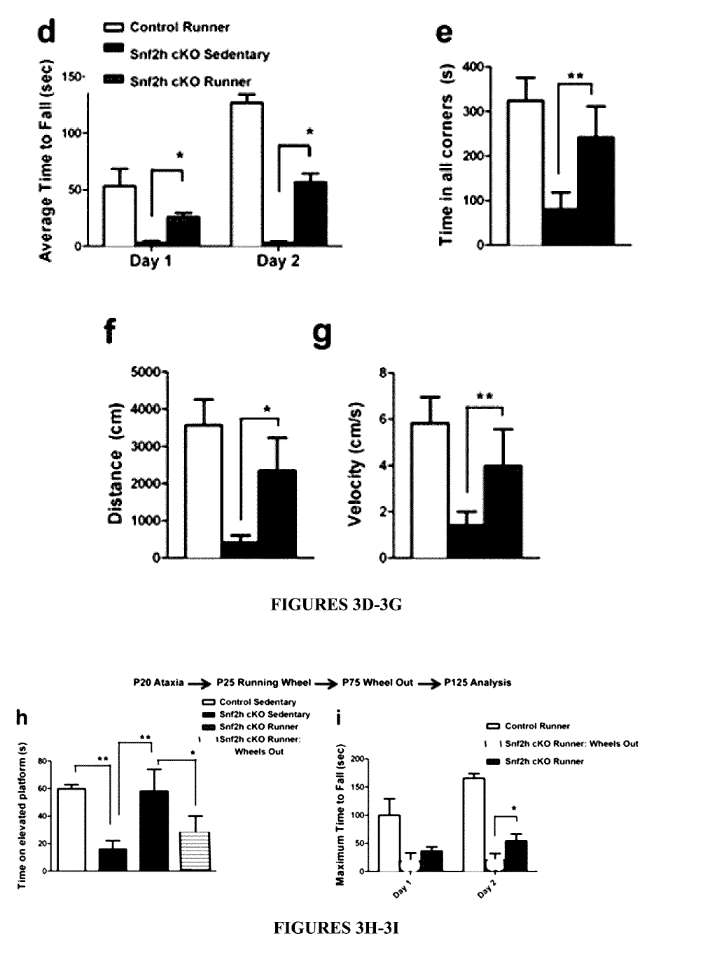

Physical exercise is known to enhance cognitive and motor functions in both healthy and disease states, has anti-depressant effects, and induces neurogenesis in the forebrain of mammals. While exercise has also been shown to delay the progression of degenerative conditions in mammalian models such as for Multiple Sclerosis (MS), Parkinson's and Alzheimer's diseases (PD, AD), the molecular basis for this benefit remains unknown. To investigate the mechanisms linking exercise to delayed neurodegeneration, mice ablated for the chromatin remodeler Snf2h in the brain that develop progressive ataxia-like signs were used. Conditional Snf2h-null mice have an abnormal gait with reduced weight gain by postnatal day 10 (P10), that progresses to classical signs of cerebellar ataxia by P20. Mice perform poorly in motor function tests at this age, continue to physically degenerate, show progressive Purkinje cell (PC) loss and die between P40-50. Morphologically, the cerebellum of Snf2h cKO mice is one-third the size of control littermates due to a severe reduction in granule neuron progenitor expansion, coupled with the progressive death of PCs. Since physical exercise can trigger both neurogenesis and oligodendrogenesis in the murine forebrain, the hypothesis whether voluntary running could be beneficial to these animals and delay disease progression was tested. Thus, these mice were provided with unlimited access to a running wheel shortly after weaning (˜P21). Strikingly, voluntary running prolonged the survival of Snflh cKO animals beyond one year of age (FIG. 3A). Running was critical for long-term survival as a cohort of mice that received the running wheel at P21-P25 but had it removed at P100 showed a progressive decline and death between P150-P200 (FIG. 3A). Along with survival, running increased weight gain in Snf2h cKO mice at a rate that was similar to wild type littermate controls (FIG. 3B). Snf2h cKO mice showed daily improvement in the distance travelled but overall averaged one half the total travelled distance of control littermates (FIG. 3C). Running also improved performance in other motor tests compared to sedentary Snf2h cKO mice (FIGS. 3D-G). Moreover, Snf2h cKO mice tested in the elevated platform and rotarod assays 50 days post-wheel removal displayed diminished performance than mice that continued to run (FIGS. 3H-I).

In more detail, FIG. 3 demonstrates that running prolongs lifespan and ameliorates motor function of mice with cerebellar ataxia. FIG. 3A depicts a Kaplan-Maier curve for Sty2h cKO (Snf2h cKO or cKO hereon) mice in sedentary (cKO-Sedentary; dotted line) or running conditions (cKO-Runner) with wheels introduced at P21 and never removed (solid line); or wheels removed at P100 (cKO-Runner, wheels out; striped line). Analysis was terminated at 1 year of age (P365). n=6-10 mice per condition. FIG. 3B depicts total body weight of cKO-Runner or Wild Type-Runner littermates (WT-Runner hereon) with wheels introduced at P25. Error bars represent ±SEM. FIG. 3C depicts total kilometers traveled for 20 days after wheels were introduced at P25. n=6, males only. FIG. 3D depicts rotarod analysis for WT-Sedentary, cKO-Sedentary, and cKO-Runner mice for 2 consecutive days 15 days post-running (wheels in at P25). *P<0.05, n=6-8 mice per condition. FIGS. 3E-G depict an open field assay for WT-Sedentary, cKO-Sedentary, and cKO-Runner mice 15 days post-running (wheels in at P25). Time in all corners, total distance traveled and velocity was averaged from 6-8 mice per genotype. *P<0.05, **P<0.01. FIGS. 3H-I depict elevated plus maze at P125 from WT-Sedentary, cKO-Sedentary, cKO-Runner, and cKO-Runner-wheels out mice (wheels in at P21-P25, wheels out at P75). *P<0.05, **P<0.01, n=6-8 mice per condition.

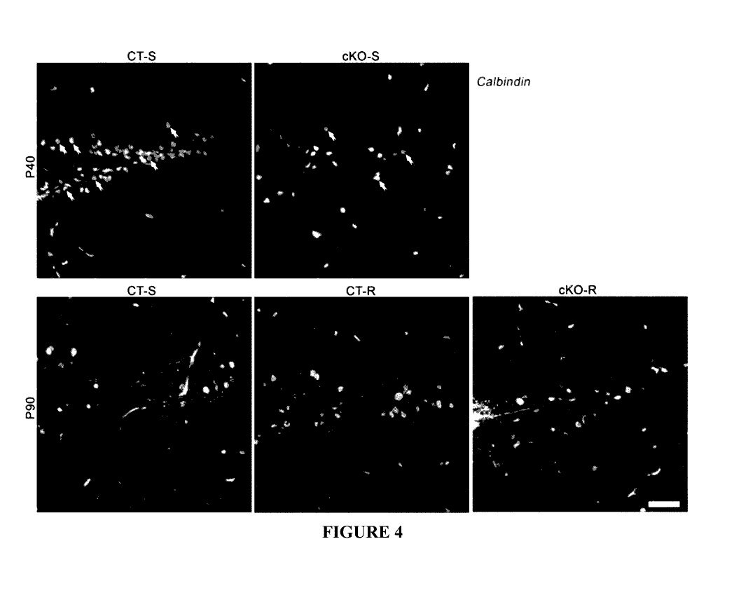

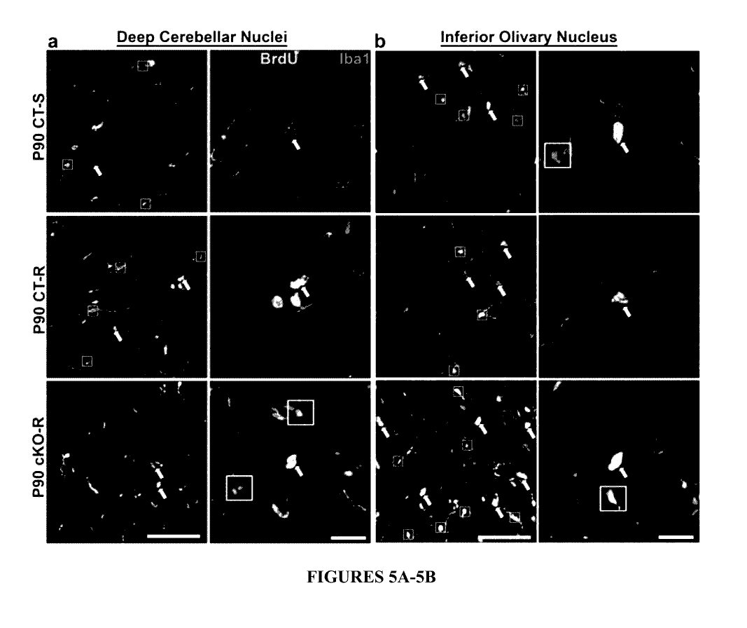

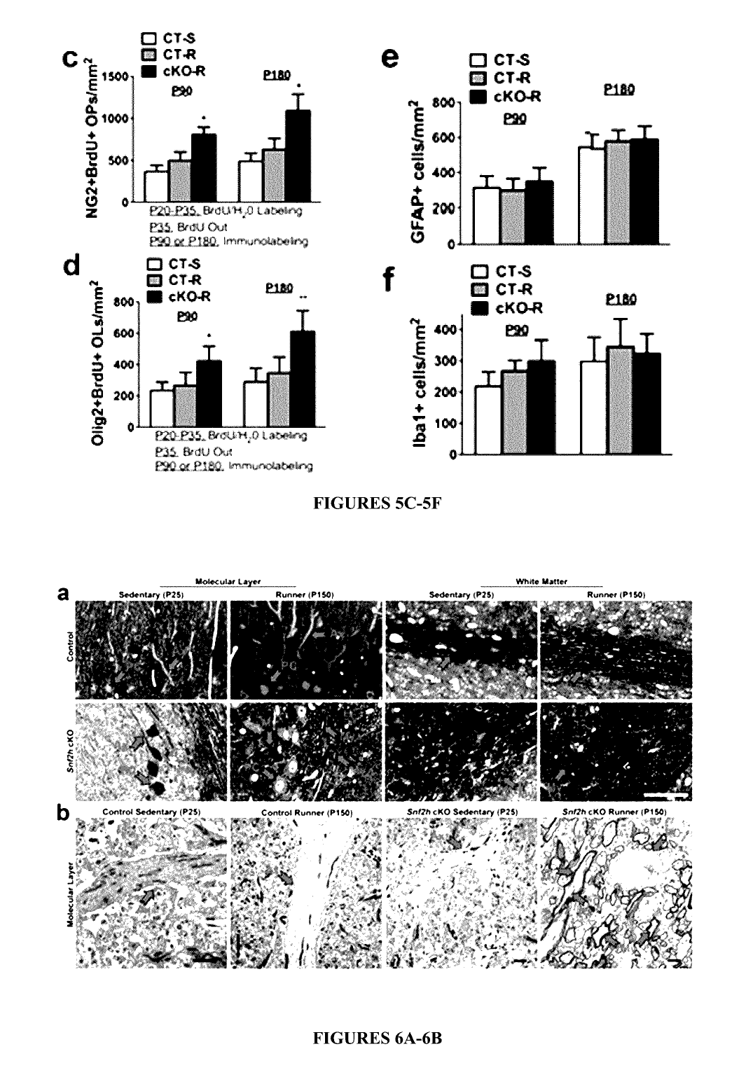

Physical exercise increases skeletal muscle metabolism by enhanced contractile and epinephrine activity that drive adaptive responses to alter whole body energy and glucose homeostasis. Amongst these responses is the secretion of autocrine and paracrine factors from the muscle, including IL-6, IL-15, IGF-1, and BDNF, amongst others, whose functions are to increase appetite, improve mood, increase mitochondrial oxidative phosphorylation, and enhance overall metabolic efficiency. BDNF has an important impact on brain function with several studies demonstrating that exercise promotes the generation of new neurons within the hippocampus, improves memory, and improves performance in aged mice. Recent studies have shown that neural activity and motor skill learning also promote oligodendrogenesis and active myelination. As such, the hypothesis that running triggers neurogenesis and/or oligodendrogenesis in hindbrain areas was assessed. BrdU was administered via the drinking water to label dividing cells between P21-P35 in wild type controls (hereon referred to as WT) and Snf2h cKO (cKO hereon) mice provided with running wheels at P21. Whole brain sagittal sections through the deep cerebellar nuclei of the cerebellum and the inferior olivary nucleus (also known as inferior olive), a major site of input into the cerebellum, were then analyzed either 55 or 145 days post-BrdU administration (P90 or 180, respectively) for BrdU+ cells co-stained with the OP (oligodendrocyte precursor) or OL (oligodendrocyte) markers NG2 and 011g2, respectively; or co-stained with Calbindin for neural lineages. As expected, prominent BrdU-labeling of neurons within the hippocampus in both WT-Runner and cKO-Runner mice relative to sedentary controls was observed. In contrast, BrdU-labeled Purkinje cells in the cerebellum of any genotype were not detected (FIG. 4, as described in more detail below). However, cKO-Runner mice showed increased BrdU+/NG2+ cell numbers within the cerebellum and the inferior olive (FIGS. 5A-C), suggesting that oligodendrogenesis may partially underlie the long-term recovery of ataxic exercising mice. Indeed, BrdU-retaining cells that co-expressed the differentiated OL marker Olig2 were significantly upregulated in cKO-Runners at P90 and P180 relative to all controls (FIG. 5D). Since neurodegenerative pathologies are also associated to increased inflammation, the total number of Iba1+ microglia or GFAP+ astrocytes within the cerebellum and inferior olive from all genotypes was assessed. This experiment revealed no changes in the total number of either Iba+ or GFAP+ cells (FIGS. 5E, F).

In more detail, FIG. 4 demonstrates that voluntary running triggers neurogenesis in the hippocampus of wild type mice. FIG. 4 shows triple immunolabeling with BrdU, Olig2 and Calbindin through the hippocampus of WT-Sedentary and cKO-Sedentary mice at P40 (top panels), and WT-Sedentary, WT-Runner and cKO-Runner mice at P90 (wheels introduced at P21; bottom panels). BrdU was administered in the drinking water from P21 to P35, and sagittal brain sections analyzed at P90, except for top panels where analysis was performed at P40. Note a mild increase in the total number of BrdU+ cells in the hippocampus of WT-Runner mice relative to WT-Sedentary mice that were scored to be neurons based on nuclear size. Note that Calbindin was not expressed in BrdU+ cells by P90, however many BrdU+ cells were also Calbindin+ at P40 (arrows). n=4 mice per condition, scale bar, 50 μm.

In more detail, FIG. 5 demonstrates that running triggers the expansion of oligodendrocyte precursors in the ataxic hindbrain. FIGS. 5A, B depict triple immunolabeling with BrdU, NG2 and Ibal through the deep cerebellar nuclei and inferior olivary nucleus of WT-Sedentary, cKO-Runner, and cKO-Sedentary mice when wheels were introduced at P21. BrdU was administered in the drinking water from P21 to P35, and sagittal brain sections analyzed at P90. Boxes highlight BrdU−, Ibal+ microglial cells, while arrows denote BrdU+, Ng2+ oligodendrocyte precursors (OPs). Note a robust increase in OPs in the inferior olivary nucleus of cKO-Runner mice. n=4 mice per condition, scale bars, 50 μm. FIGS. SC-F depict total cell counts from 1mm²×100um³ confocal Z-stacks through the inferior olivary nucleus of WT-Sedentary, cKO-Sedentary, and cKO-Runner mice treated with BrdU between P21-P35 (wheels in at P21) and analyzed 55 days post-BrdU removal (P90); or 145 days post-BrdU removal (P180). Error bars represent ±SEM. *P<0.05, **P<0.01, n=4 mice per condition.

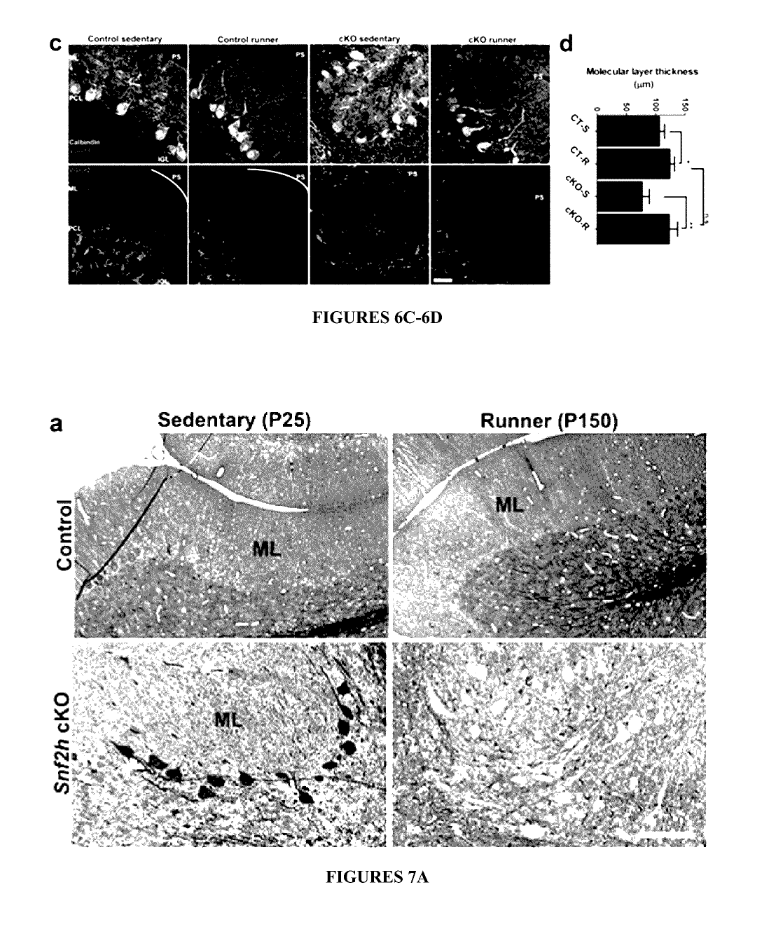

To assess whether the increased number of Ng2+ and Olig2+ OPs and OLs resulted in increased axonal myelination, toluidine blue-stained and ultrathin transmission electron microscopy (TEM) images from the molecular layer and the white matter of the cerebellum were obtained from WT and cKO mice 125 days post-running (wheels introduced at P25), and from P25 WT and cKO sedentary mice as controls. Strikingly, myelination in the molecular layer of cKO-Runner mice that was not present in WT-Sedentary, WT-Runner, or cKO-Sedentary mice was observed (FIGS. 6A, B). As control, the white matter from all genotypes showed comparable myelination (FIG. 6A).

In more detail, FIG. 6 demonstrates that running triggers de novo myelination and enhanced Purkinje cell arborization through the ataxic cerebellum. FIG. 6A depicts toluidine blue staining through the cerebellar vermis (molecular layer) and the deep cerebellar nuclei (white matter) of WT-Sedentary and cKO-Sedentary mice at P25, and WT-Runner and cKO-Runner mice at P150 (wheels in at P25). Note the massive appearance of myelin rings through the cKO-Runner cerebellum (arrows). PC, Purkinje cell, n=4 mice per genotype, scale bars, 100 μm. FIG. 6B depicts transmission electron microscopy (TEM) analysis through axons within the molecular layer of WT and cKO-Sedentary mice at P25, and WT and cKO-Runner mice at P150 (wheels in at P25). Note the robust de novo myelination through axonal processes in cKO-R cerebella. Ax, axon. n=4 mice per genotype, scale bars, 2 μm. FIG. 6C depicts triple immunolabeling through the molecular layer of WT-Sedentary, Sedentary, cKO-Sedentary, WT-Runner, and cKO-Runner at P40 when wheels were introduced at P25 with the excitatory synaptic markers VGlut1, VGlut2, and Calbindin. DAPI stains all nuclei. Note the increased arborization of PC dendritic trees in both WT-R and cKO-R cerebella. n=4 per genotype, scale bars, 20₁1m. FIG. 6D depicts quantitation of the molecular layer thickness of WT-Sedentary, cKO-Sedentary, WT-Runner, and cKO-Runner at P40 mice when wheels were introduced at P25. Error bars represent ±SEM. *P<0.05, **P<0.01, n.s.=not significant, n=4 per condition.

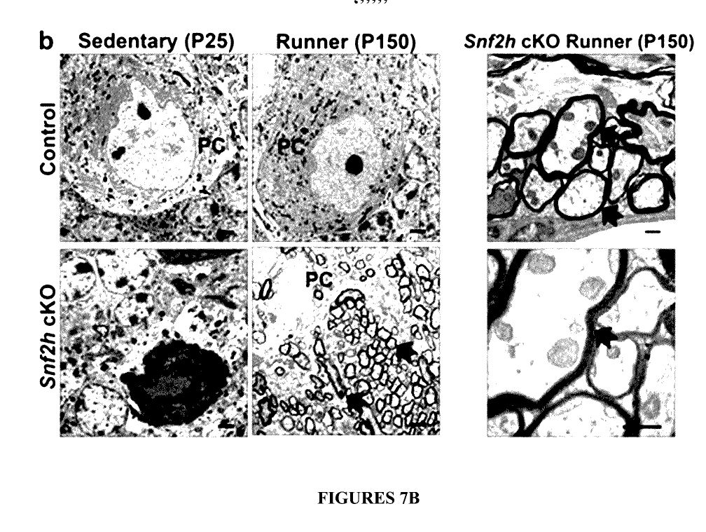

Myelination is not typically present within the molecular layer of the cerebellum and due to the disorganization of the mutant cerebellum, it was not possible to determine if the myelination was present on axons of PCs projecting from a different plane, or derived from the parallel and climbing fibers of granule or inferior olivary neurons, respectively (FIG. 7, as described in more detail below). Regardless, it was hypothesized that increased myelination protects and strengthens the function of the existing neurons in the cerebellum of mutant mice. Since increased myelination confers improved neuronal function and because running activity was shown to improve PC dendritic arborization, the thickness of the molecular layer was examined. Indeed, a small but significant increase in the thickness of the molecular layer in both WT-Runner and cKO-Runner mice versus WT-Sedentary and cKO-Sedentary animals was observed (FIG. 6C, D). These results are further supported as increased Purkinje cell dendritic growth has been previously attributed to running activity in wild type rodents.

In more detail, FIG. 7 demonstrates that voluntary running triggers de novo myelination in the cerebellar vermis of ataxic mice. FIG. 7A depicts toluidine blue staining through the cerebellar vermis of WT-Sedentary and cKO-Sedentary mice at P25 (left panels), and WT-Runner and cKO-Runner mice at P150 (wheels in at P25; right panels). ML=molecular layer, PC=Purkinje cell, Ax=axon, My=myelin, n=4 mice per genotype, scale bar, 100 μm. FIG. 7B depicts transmission electron microscopy (TEM) analysis through axons within the cerebellar vermis of WT-Sedentary and cKO-Sedentary mice at P25, and WT-Runner and cKO-Runner mice at P150 (wheels introduced at P25). Note the robust de novo myelination through axonal processes in cKO-R cerebella (arrows). PC, Purkinje cell, n=4 mice per genotype, scale bars, 2 μm, except for bottom rightmost panel where scale bar, 500 nm.

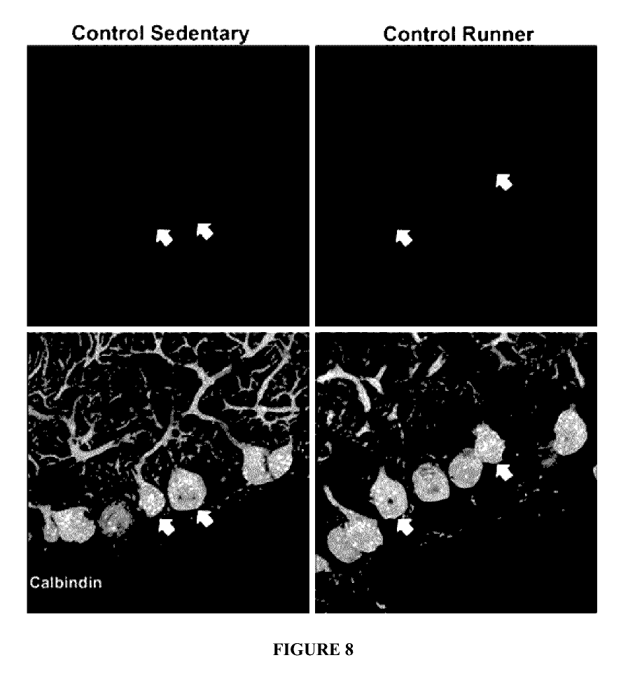

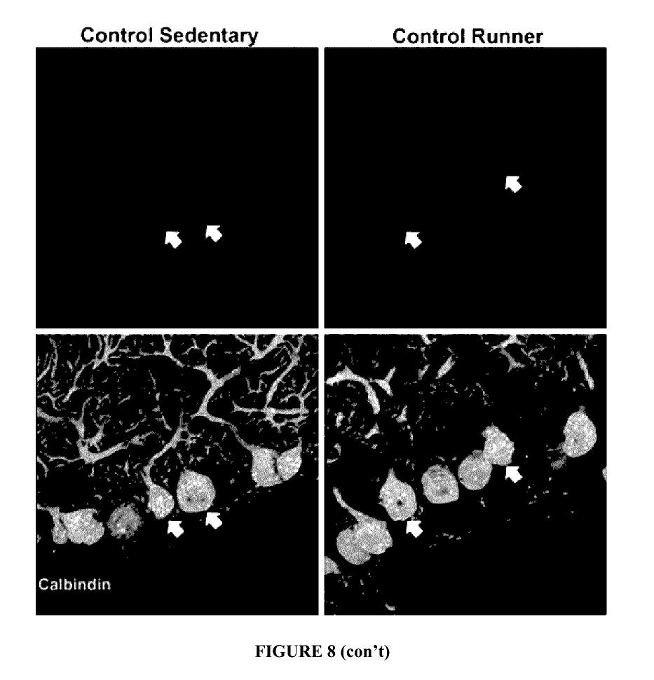

To attribute this to an actual neuronal effect, WT and cKO Sedentary or Running (15 days post-running; wheels in at P25) mice were co-labeled with the serotonin transporter (Slc4a6), and Calbindin, a marker of PCs. Increased Slc4a6 puncta was observed within the PC soma of cKO-R mice relative to all control genotypes, suggesting that running enhanced the activity and survival of existing mutant PCs (FIG. 8, as described in more detail below).

In more detail, FIG. 8 demonstrates that voluntary running upregulates serotonin transporter synthesis in ataxic Purkinje neurons. FIG. 8 depicts co-immunolabeling with Slc6a4 (serotonin transporter) and Calbindin through the cerebellar vermis of wild type and Snf2h cKO mice in sedentary and running conditions at P41 (wheels introduced at P21). DAPI (top panels) stains all nuclei. Note a robust increase in Slc6a4 levels within PC nuclei (arrows) in Snf2h cKO runner cerebella. n=4 mice per genotype, scale bars, 50 μm.

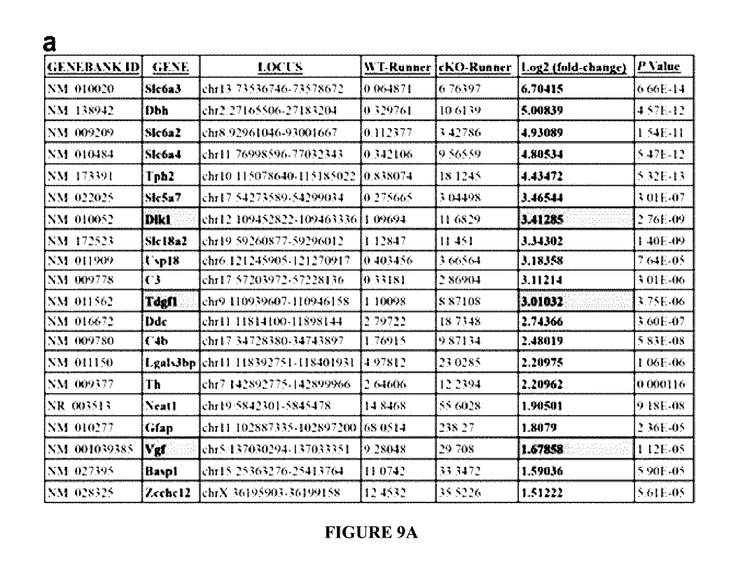

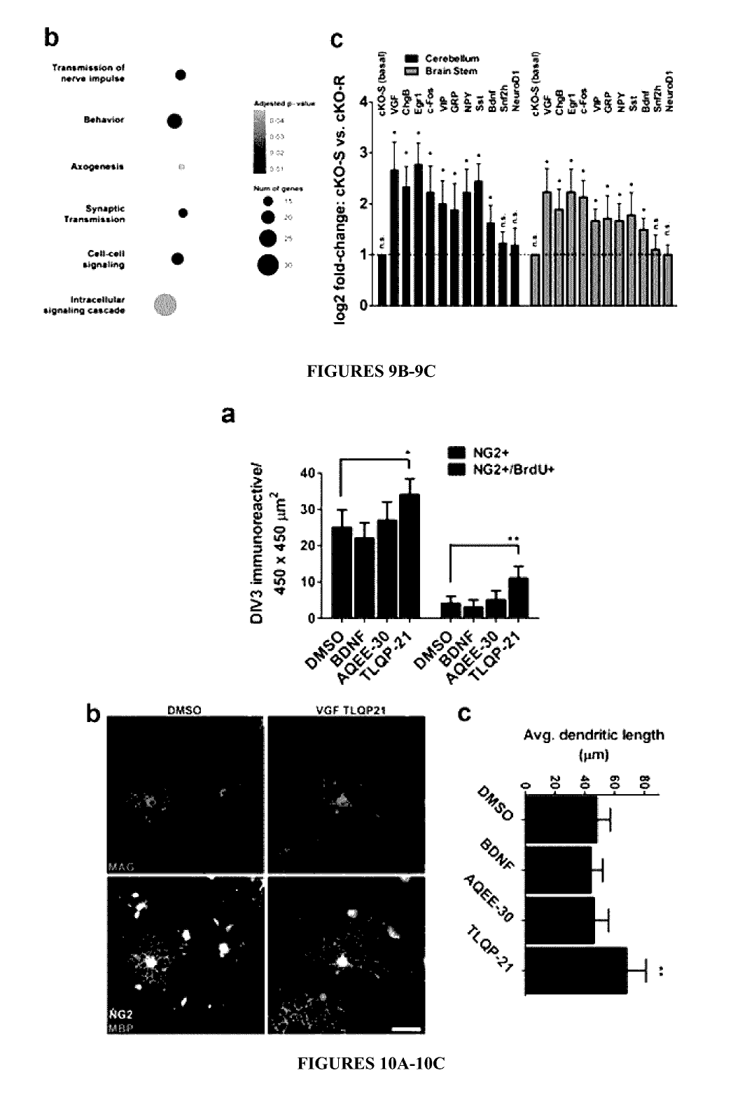

Concomitant to the cellular studies, RNA-Sequencing experiments were performed to identify the molecular correlates contributing to the exercise-induced rescue of Snf2h cKO mice. A four-way comparative analysis was performed between samples sequenced from WT-Sedentary, WT-Runner, cKO-Sedentary and cKO-Runner cerebella, identifying 2290 upregulated and 1321 downregulated genes in the cKO-R vs. WT-R analysis (FIG. 9A). Gene Ontology (GO) analyses were consistent with the findings, namely that genes involved in activity-dependent synaptic transmission were increased (FIG. 9A, B). In addition, an increase in growth factors and the exercise-induced neuropeptide precursor VGF (non-acronymic; also known as nerve-growth factor inducible) was observed (FIG. 9A). The results were then validated both as significantly upregulated in cKO-Runner vs. cKO-Sedentary cerebellum and brain stem tissue with TaqMan-based quantitative PCR (FIG. 9C).

In more detail, FIG. 9 demonstrates that running upregulates synaptic transmission and growth factor synthesis in the ataxic cerebellum and brain stem. FIG. 9A demonstrates that RNA-Seq reveals the top 20 upregulated hits by differential expression (DE) analysis between cKO-Runner vs. WT-Runner cerebella after 15 days of running (wheels in at P25). Note the high number of genes involved in activity-dependent synaptic transmission (light shading) and growth factor synthesis (dark shading). P<0.05, n=2 paired-end libraries per genotype. See FIG. 9A for DE analyses between WT-Runner and cKO-Runner mice. FIG. 9B depicts Gene Ontology (GO) biological processes enriched in upregulated gene sets from cKO-Runner vs. WT-Runner cerebella. Selected genes were analyzed by DAVID for enriched GO terms and shown with Benjamini adjusted P-values of <0.05. FIG. 9C depicts TaqMan probe-based qPCR validation for selected growth factors and immediate early genes between cKO-Runner vs. cKO-Sedentary cerebella and brain stems. L32 was used as internal control. *P<0.05, n.s.=not significant, n=4 per condition.

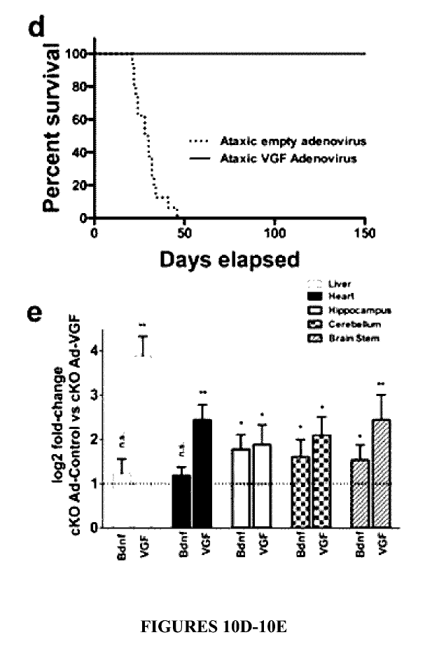

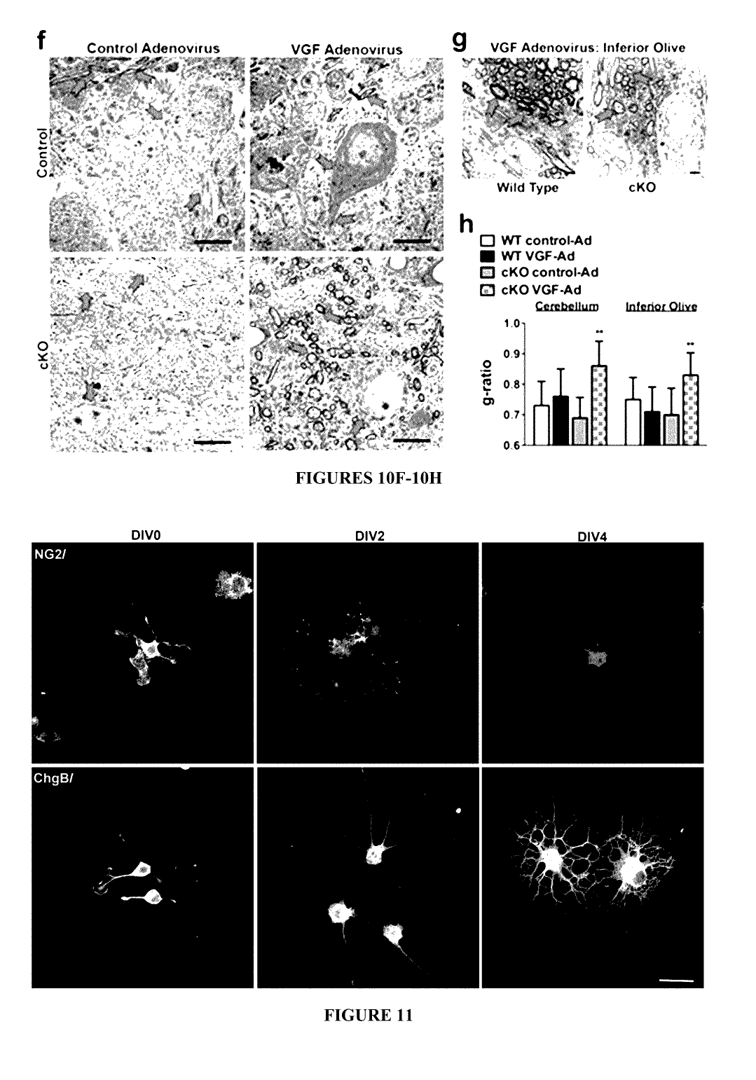

The growth factor VGF was of particular interest because it is reported to be highly expressed in OPs. Furthermore, its upregulation upon exercise activates the anti-depressant response in mammals, and maintains BDNF expression in a VGF-BDNF positive feedback loop. Hence, it was determined whether VGF peptides could stimulate the growth and differentiation of homogenous preparations of primary mouse OPs. VGF is cleaved to produce numerous C-terminal bioactive peptides including TLQP-62, TLQP-21, and AQEE-30, amongst others. As such, oligodendrocyte precursor (OP) cultures were treated with well-characterized peptides that trigger BDNF production TLQP-21 (3 μM), AQEE-30 (3 μM), BDNF (50 ng/μl), or DMSO along with the proliferation marker BrdU and then analyzed 48 hours later by scoring the number of NG2+/BrdU+ cells. In parallel, the total number of NG2+ cells was counted to assess for endogenous proliferation. Interestingly, a significant increase in the total number of NG2+ cells was observed, and in the total number of proliferating Ng2+/BrdU+ OPs when treated with the TLQP-21 VGF peptide. In contrast, no significant differences were observed in any of the other treatments (FIG. 10A). Differentiation of the cultures with the mature oligodendrocyte (OL) markers myelin associated glycoprotein (MAG) and myelin basic protein (MBP) was next assessed, where TLQP-21 also increased the average oligodendrocyte dendritic length suggesting that the peptide can promote both OP proliferation and their subsequent differentiation into OLs (FIG. 10B,C). Indeed, VGF protein levels are robust early in the OP differentiation cascade (FIG. 11). Taken together, these results raised the possibility that VGF is critical to the long-term survival of running Snf2h-null mice.

In more detail, FIG. 11 demonstrates that the g,ranin family proteins Chromogranin-B and VGF are robustly expressed in developing wild type OPs. Mouse OPs were isolated from the cortices of PO WT neonates by expansion within mixed glial culture for 8 days, purified, and subsequently differentiated as an OP-enriched culture. Top panels: cells were co-immunolabeled with NG2 and VGF at 0 days, 2 days or 4 days after purification. Bottom panels: cells were co-immunolabeled with Chromogranin-B and VGF at the indicated time points. Scale bar, 10 μm.

To determine if VGF was sufficient to rescue the lifespan of cKO mice, adenoviral (Ad)-vectors expressing full-length mouse VGF protein were generated. Control and sedentary cKO mice received tail vein injections at ˜P21 (1×10¹² viral particles per kilogram), and their survival was recorded. Strikingly, cKO mice that received the Ad-VGF injections were still alive and healthy at P150 (time of analysis) whereas uninjected cKO or Ad-control injected cKO mice perished between P25 and P45 using both C57B1/6 and FVB/N backcrossed cKO strains (FIG. 10D). Using qPCR to compare transcript levels between cKO Ad-control and cKO Ad-VGF, significant VGF mRNA upregulation in the heart, hippocampus, cerebellum and brain stem, with highest levels in the liver, the primary site of adenoviral transduction were detected (FIG. 10E). However, upregulation of the Bdnf transcript was only detected in the brain (hippocampus, cerebellum and brain stem), and not in any of the other tissues analyzed (FIG. 10E). Importantly, TEM images demonstrated increased myelination in the molecular layer of the cerebellum and the inferior olive of cKO Ad-VGF treated animals versus WT Ad-VGF or cKO Ad-control treated mice, suggesting that increasing cerebellar myelination was critical for the survival of the sedentary cKO mice (FIG. 10F-H). Of note, some additional, but non-significant myelination in Ad-VGF injected WT animals was observed, but it was minimal compared to the rescued Ad-VGF treated sedentary cKO mice.

In more detail, FIG. 10 demonstrates that VGF TLQP-21 stimulates OP expansion and differentiation, while full-length VGF overexpression prolongs lifespan and triggers de novo myelination in the ataxic cerebellum. Mouse OPs were isolated from the cortices of PO WT neonates by expansion within mixed glial culture for 8 days, purified, and subsequently differentiated as an OP-enriched culture. At DIV1 post-purification, cells were treated with DMSO, BDNF, VGF AQEE-30 or VGF TLQP-21 at 3 uM and allowed to grow for an additional 48 hrs. FIG. 10A shows, in a parallel experiment, cells were further supplemented with BrdU (20 nM) 6 hrs after peptide or DMSO treatment and BrdU+, NG2+ or total NG2+ cells counted in 450×450 μm² bins 42-hrs post-BrdU treatment. FIG. 10B shows that at DIV3 post-purification, cells were triple immunolabeled with NG2, a marker of OPs; MAG, a marker of differentiating OLs; and MEP, a marker of myelin-associated glycoprotein. DAPI stains all nuclei. Note the enlarged processes in OLs treated with VGF TLQP-21 vs. DMSO controls. Scale bar, 10 m. FIG. 10C depicts quantitation of the average dendritic length 48 hrs post-peptide treatment. **P<0.01, n=4 independent coverslips per condition. FIG. 100 shows that Early region 1 (E1)/E3-deleted adenoviral vectors driving an empty cassette (Ad-Control) or full-length mouse VGF (Ad-VGF) under the regulation of the human CMV promoter were delivered via tail-injection at P21 in cKO-Sedentary mice. Kaplan-Maier curves highlight the extended lifespan of cKO-Sedentary mice treated with Ad-VGF viral particles (solid line) relative to cKO-Sedentary mice treated with Ad-Empty control (dotted line). Experiments were terminated at P150 for tissue analysis. FIG. 10E depicts Taq-Man probe-based qPCR expression analysis for BDNF and VGF in selected tissues 30 days after viral delivery (˜P51). L32 was used as internal control. *P<0.05, **P<0.01, n.s.=not significant, n=4 per genotype. FIGS. 10F-G depicts TEM analysis through the molecular layer of the cerebellum (FIG. 10F) or the inferior olive (FIG. 10G) of WT-Sedentary and cKO-Sedentary mice at P60 treated with empty Ad-CT or Ad-VGF vectors at ˜P21. Note the robust myelination in the cerebellum of Ad-VGF treated cKO mice. PC=Purkinje cell; Ax=axon; My=myelin; n=6 mice per genotype, scale bars, 10 μm. FIG. 10H depicts g-ratios of axons within the molecular layer of the cerebellum and the inferior olive of WT-Sedentary and cKO-Sedentary mice at P60 treated with empty Ad-CT or Ad-VGF vectors at ˜P21. **P<0.01, ˜50 axons were scored from 6 independent mice per genotype.

Taken together, the results suggest that damaged cerebellar neural circuits can be substantially rescued by increased myelination, which remarkably was observed in a voluntary running-induced manner based on an endogenous mechanism of brain repair. In a normally functioning cerebellum additional myelination is not as significant as in the pathological state, suggesting that exercise promotes a repair mechanism that counteracts a neurodegenerative process. The results provide strong evidence that VGF has the ability to stimulate myelination, revert neural damage, and prolong lifespan in this disease model. These findings thereby illuminate a novel pathway to potentially treat other neuronal and oligodendroglia-associated pathologies of the central nervous system.

### Example 2

### Running Stimulates VGF Production in the Heart

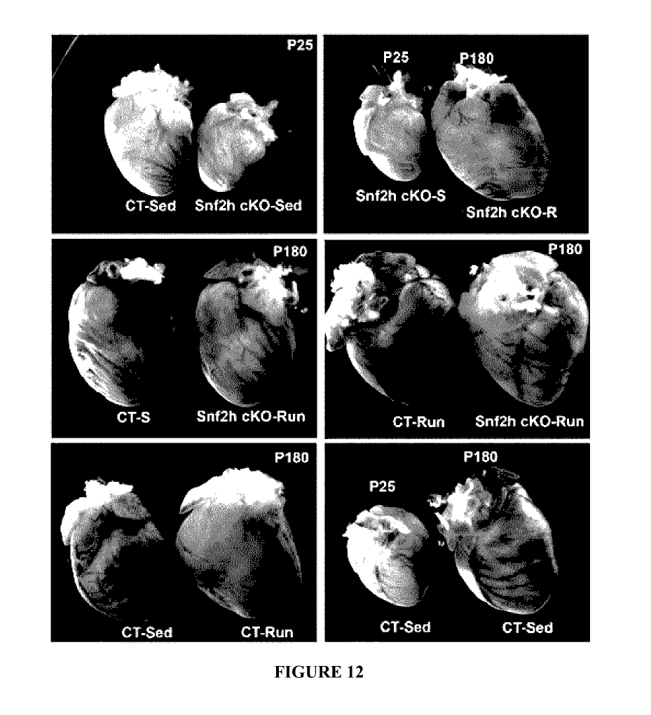

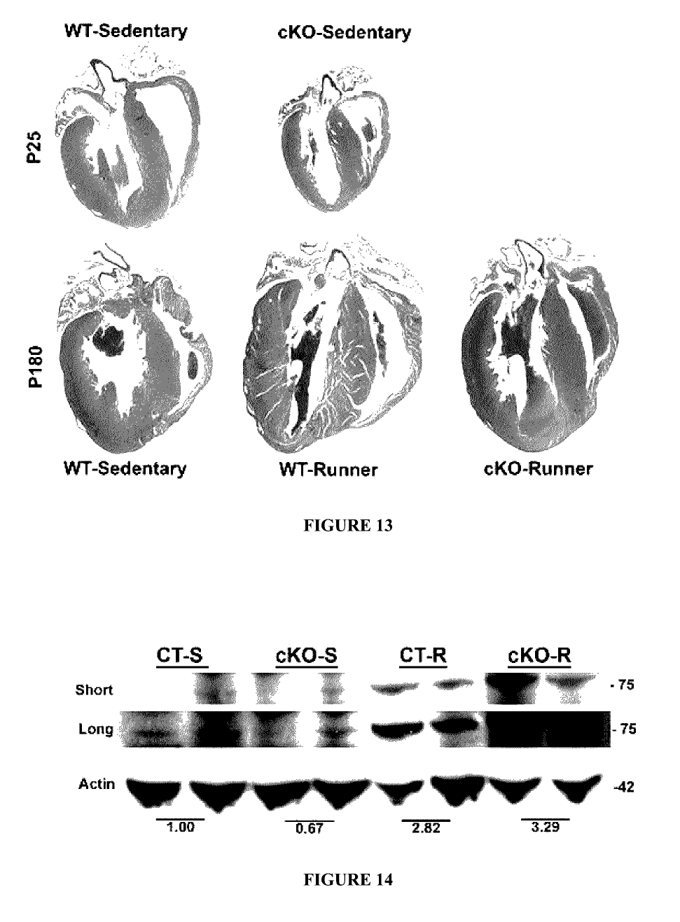

In order to characterize the effects of running on cardiac remodeling and VGF plasma levels, the hearts of Sq/2h cKO mice in sedentary conditions relative to Sq/2h cKO runners were analyzed, as well as wild type controls in both sedentary and running conditions after 160 days of running (wheels in at P20). First, dissected whole hearts were dissected and whole mount imaging was performed. Indeed, FIG. 12 depicts whole mount images from hearts dissected at P25 or P180 from wild type and Snf2h cKO-mice in sedentary and running paradigms. S or Sed, sedentary; R or Run, runner; P, postnatal day. Note the normalization in heart size in Snf212 cKO running mice compared to wild type running controls. Additionally, histology was performed in these same hearts. FIG. 13 depicts coronal sections from hearts dissected at P25 or P180 from wild type and Snf2h cKO mice in sedentary and running paradigms stained with Eosin and Haematoxylin. Lastly, to assess VGF levels in the circulating plasma, plasma was isolated from Snf2h cKO and control mice in running and sedentary conditions after 15 days of running (wheels in at P25). Strikingly, FIG. 14 depicts Western blotting from plasma obtained from P40 wild type and Snflh cKO mice in sedentary and running paradigms (wheels in at P25 for runner) with antibodies against full-length VGF (top panels, short and long exposures shown); and Actin (bottom panels) as loading control. Each lane represents a pool of 2 plasma samples from mice of the same cohort and genotype. S, sedentary; R, runner. Numbers on the right represent lcDa (kilodaltons). Densitometry values are presented in the bottom normalized to CT-S levels.

### Example 3

### Administration of VGF Protein to Treat Multiple Sclerosis

A patient presents at a clinic with symptoms of multiple sclerosis. The patient undergoes an MRI and numerous brain lesions are detected. The patient is also positive for oligoclonal bands. The patient is diagnosed with multiple sclerosis. A healthcare practitioner administers 1 μg to 10 mg of VGF peptide, TLQP-21, intravenously. A follow-up MRI is performed over the following months. The patient has significantly fewer brain lesions as compared to the previous MRI of the patient. The patient's symptoms also improve over the following months.

### Example 4

### Administration of VGF Protein to Treat an Aortic Aneurysm

A patient presents at a clinic with symptoms of an aortic aneurysm. The patient undergoes an MRI and the patient is diagnosed with an aortic aneurysm. A healthcare practitioner administers 1 μg to 10 mg of VGF peptide, TLQP-21, intravenously. A follow-up MRI is performed over the following months. The MRI demonstrates that the aortic aneurysm is responsive to TLQP-21 therapy and the size of the aortic aneurysm decreases.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the invention has been described with reference to the above examples, it will be understood that modifications and variations are encompassed within the spirit and scope of the invention. Accordingly, the invention is limited only by the following claims.

What is claimed is:

1. A method of treating a neurodegenerative disease comprising delivering to a subject having or at risk of having a neurodegenerative disease, a nucleic acid molecule encoding exogenous VGF or a functional peptide thereof, thereby treating the neurodegenerative disease.

2. The method of claim 1, wherein said nucleic acid molecule is delivered by an adeno-associated viral (AAV) vector, a retroviral vector, or an adenoviral vector.

3. The method of claim 2, wherein said AAV vector is AAV-2 or AAV-9.

4. The method of claim 1, wherein said subject is a human.

5. The method of claim 1, wherein said delivering to a subject comprises intravenous, intranasal, intrathecal, or oral administration.

6. The method of claim 1, wherein said delivering to said subject comprises intracranial administration.

7. The method of claim 1, wherein said exogenous VGF or said peptide thereof is expressed in the brain of said subject.

8. The method of claim 7, wherein said exogenous VGF or said peptide thereof is expressed in glial cells.

9. The method of claim 8, wherein said glial cells are oligodendrocytes or oligodendrocyte precursors.

10. The method of claim 1, wherein said neurodegenerative disease is a demyelinating disease.

11. The method of claim 10, wherein said demyelinating disease is cerebellar ataxia, multiple sclerosis, Alzheimer's disease, amyotrophic lateral sclerosis, or Friedreich's ataxia.

12. The method of claim 1, wherein said treating results in remyelination, de novo myelination or hypermyelination.

13. The method of claim 1, wherein said exogenous peptide is TLQP-21, TLQP-62, AQEE-30, NERP-1, NERP-2, LENY-10, RSQE-9, VGF₄₄₃₋₅₈₈ Or VGF₄₋₂₄₀.

14. The method of claim 1, wherein said exogenous VGF or said peptide thereof is expressed at a higher level in said subject relative to said subject prior to delivery of said nucleic acid molecule.

15. The method of claim 1, wherein said exogenous VGF or said peptide thereof comprises at least 50% amino acid sequence homology to a native VGF or a peptide thereof

16. The method of claim 1, wherein said exogenous VGF or said peptide thereof comprises at least 50% nucleic acid sequence homology to a native VGF or a peptide thereof.

17. The method of claim 16, wherein said VGF is human.

18. The method of claim 10, wherein said demyelinating disease is diagnosed by identifying at least one brain lesion in said subject by magnetic resonance imaging.

19. The method of claim 18, wherein said subject has fewer brain lesions after said delivering than before said delivering.

20. A method of treating a neurodegenerative disease comprising delivering exogenous VGF protein or a peptide thereof to a subject having or at risk of having said neurodegenerative disease, thereby treating the disease.

21. The method of claim 20, wherein said delivering comprises intravenous, intranasal, intrathecal or oral administration.

22. The method of claim 20, wherein said neurodegenerative disease is a demyelinating disease.

23. The method of claim 20, wherein said delivering comprises intracranial administration.

24. The method of claim 20, wherein said demyelinating disease is cerebellar ataxia, multiple sclerosis, Alzheimer's disease, amyotrophic lateral sclerosis, or Friedreich's ataxia.

25. The method of claim 20, wherein said exogenous peptide is TLQP-21, TLQP-62, AQEE-30, NERP-1, NERP-2, LENY-10, RSQE-9, VGF₄₄₃₋₅₈₈ or VGF₄₋₂₄₀.

26. The method of claim 20, wherein said exogenous VGF protein or peptide thereof comprises at least 50% amino acid sequence homology to a native VGF protein or a peptide thereof.

27. The method of claim 26, wherein said native VGF protein is human.

28. The method of claim 20, wherein said subject is a human.

29. The method of claim 20, wherein said delivering results in remyelination, de novo myelination or hypermyelination.

30. The method of claim 22, wherein said demyelinating disease is diagnosed by identifying at least one brain lesion in said subject by magnetic resonance imaging.

31. The method of claim 30, wherein said subject has less brain lesions after said delivering than before said delivering.

32. A method for detecting a level of VGF protein or a peptide thereof in a plasma sample of a subject comprising comparing said level of said VGF protein or said peptide thereof to that of a healthy control.

33. The method of claim 32, wherein said level of VGF protein or said VGF peptide thereof is at least 2-fold lower in said subject than said healthy control.

34. The method of claim 32, wherein said level of VGF protein or said VGF peptide thereof is at least 10-fold lower in said subject than said healthy control.

35. The method of claim 32, wherein said detecting comprises mass spectrometry.

36. The method of claim 32, wherein said detecting comprises an ELISA assay.

37. A method of inducing remyelination, de novo myelination or hypermyelination in a subject, comprising administering (i) a nucleic acid molecule encoding an exogenous VGF or peptide thereof to a subject, wherein said exogenous VGF or peptide thereof is expressed in the subject, or (ii) an exogenous VGF protein or peptide thereof; wherein said administering induces remyelination, de novo myelination or hypermyelination in said subject.

38. The method of claim 37, wherein said subject is suffering from a neurodegenerative disease.

39. The method of claim 38, wherein said neurodegenerative disease is a demyelinating disease.

40. The method of claim 39, wherein said demyelinating disease is cerebellar ataxia, multiple sclerosis, Alzheimer's disease, amyotrophic lateral sclerosis, or Friedreich's ataxia.

41. The method of claim 39, wherein said demyelinating disease is diagnosed by identifying at least one brain lesion in said subject by magnetic resonance imaging.

42. The method of claim 41, wherein said subject has less brain lesions after said than before said administering.

43. The method of claim 42, wherein said exogenous VGF or peptide thereof is expressed in the brain.

44. The method of claim 43, wherein said exogenous VGF or peptide thereof is expressed in glial cells.

45. The method of claim 44, wherein said glial cells are oligodendrocytes or oligodendrocyte precursors.

46. A method of treating a neurodegenerative disease comprising delivering to a subject having or at risk of having a neurodegenerative disease, a CRISPR-Cas9 complex to mediate transcriptional activation at the endogenous VGF locus, thereby treating the neurodegenerative disease.
